(19) United States
(12) Patent Application Publication
(10) Pub. No.: US 2026/0000706 A1
(43) Pub. Date: Jan. 1, 2026

(54) METHODS OF TREATING

(71) Applicant: Ariel Scientific Innovations Ltd., Ariel (IL)

(72) Inventor: Danny BARANES, Ramat HaSharon (IL)

(73) Assignee: Ariel Scientific Innovations Ltd., Ariel (IL)

(21) Appl. No.: 19/321,368

(22) Filed: Sep. 8, 2025

Related U.S. Application Data
(63) Continuation of application No. PCT/IL2024/050246, filed on Mar. 7, 2024.
(60) Provisional application No. 63/450,691, filed on Mar. 8, 2023.

(51) Int. Cl. A61K 33/10; A61K 9/00; A61K 9/14; A61K 31/194; A61K 31/198; A61K 35/614; A61K 47/10; A61K 47/36; A61K 47/42; A61P 1/02; A61P 17/02; A61P 17/04; A61P 21/00; A61P 29/00; A61P 35/00

(52) U.S. Cl. CPC A61K 33/10; A61K 9/0014; A61K 9/143; A61K 31/194; A61K 31/198; A61K 35/614; A61K 47/10; A61K 47/36; A61K 47/42; A61P 1/02; A61P 17/02; A61P 17/04; A61P 21/00; A61P 29/00; A61P 35/00

(57) ABSTRACT

Provided herewith are treatment methods and generally therapeutic applications which make use of a composite that comprises a calcium carbonate-containing material, and an associating moiety being associated with said calcium carbonate-containing material.

### RELATED APPLICATIONS

This application is a Continuation of PCT Patent Application No. PCT/IL2024/050246 having International filing date of Mar. 7, 2024, which claims the benefit of priority under 35 USC § 119 (e) of U.S. Provisional Patent Application No. 63/450,691 filed on Mar. 8, 2023. The contents of the above applications are all incorporated by reference as if fully set forth herein in their entirety.

### FIELD AND BACKGROUND OF THE INVENTION

The present invention, in some embodiments thereof, relates to methods of treating.

The need to find new medications for multiple diseases arises from several factors, including the emergence of new diseases, the limitations of existing treatments, and the increasing prevalence of certain diseases.

One of the primary drivers for new medications includes the limitations of existing treatments. Many diseases have treatments that are effective to a certain extent but may have significant side effects or may not work for all patients. In addition, some diseases, such glioblastoma disease, have no effective treatments at all. New medications are needed to overcome these limitations and provide better options for patients

The increasing prevalence of certain diseases, such as diabetes, obesity, and cancer, also underscores the need for new medications. As the global population grows and ages, these diseases are becoming more common and placing a significant burden on healthcare systems worldwide. New medications are needed to address these growing health challenges and improve patient outcomes.

### RELATED BACKGROUND ART

WO2017/077539
WO2021/050244

### SUMMARY OF THE INVENTION

According to an aspect of some embodiments of the present invention there is provided a method of treating an aphtha in a subject in need thereof, the method comprising administering to the subject an effective amount of a composite material comprising a calcium carbonate-containing material, and an associating moiety being associated with the calcium carbonate-containing material, thereby treating aphtha in the subject.

According to an aspect of some embodiments of the present invention there is provided a composite material comprising an effective amount of a calcium carbonate-containing material, and an associating moiety being associated with the calcium carbonate-containing material for use in treating aphtha in a subject in need thereof.

According to an aspect of some embodiments of the present invention there is provided a method of treating a chemotherapy-induced wound in a subject in need thereof, the method comprising administering to the subject an effective amount of a composite material comprising a calcium carbonate-containing material, and an associating moiety being associated with the calcium carbonate-containing material, thereby treating the chemotherapy-induced wound in the subject.

According to an aspect of some embodiments of the present invention there is provided a composite material comprising an effective amount of calcium carbonate-containing material, and an associating moiety being associated with the calcium carbonate-containing material for use in treating a chemotherapy-induced wound in a subject in need thereof.

According to an aspect of some embodiments of the present invention there is provided a method of treating acne in a subject in need thereof, the method comprising administering to the subject an effective amount of a composite material comprising a calcium carbonate-containing material, and an associating moiety being associated with the calcium carbonate-containing material, thereby treating the acne in the subject.

According to an aspect of some embodiments of the present invention there is provided a composite material comprising an effective amount of calcium carbonate-containing material, and an associating moiety being associated with the calcium carbonate-containing material for use in treating acne in a subject in need thereof.

According to an aspect of some embodiments of the present invention there is provided a method of treating hemorrhoids in a subject in need thereof, the method comprising administering to the subject an effective amount of a composite material comprising a calcium carbonate-containing material, and an associating moiety being associated with the calcium carbonate-containing material, thereby treating hemorrhoids in the subject.

According to an aspect of some embodiments of the present invention there is provided a composite material comprising an effective amount of calcium carbonate-containing material, and an associating moiety being associated with the calcium carbonate-containing material for use in treating hemorrhoids in a subject in need thereof.

According to an aspect of some embodiments of the present invention there is provided a method of treating a fissure in a subject in need thereof, the method comprising administering to the subject an effective amount of a composite material comprising a calcium carbonate-containing material, and an associating moiety being associated with the calcium carbonate-containing material, thereby treating the fissure in the subject.

According to an aspect of some embodiments of the present invention there is provided a composite material comprising an effective amount of calcium carbonate-containing material, and an associating moiety being associated with the calcium carbonate-containing material for use in treating a fissure in a subject in need thereof.

According to an aspect of some embodiments of the present invention there is provided a method of treating itching in a subject in need thereof, the method comprising administering to the subject an effective amount of a composite material comprising a calcium carbonate-containing material, and an associating moiety being associated with the calcium carbonate-containing material, thereby treating itching in the subject.

According to an aspect of some embodiments of the present invention there is provided a composite material comprising an effective amount of calcium carbonate-containing material, and an associating moiety being associated with the calcium carbonate-containing material for use in treating itching in a subject in need thereof.

According to an aspect of some embodiments of the present invention there is provided a method of treating a chemical-induced burn or a heat-induced burn in a subject in need thereof, the method comprising administering to the subject an effective amount of a composite material comprising a calcium carbonate-containing material, and an associating moiety being associated with the calcium carbonate-containing material, thereby treating the chemical-induced burn or a heat-induced burn in the subject.

According to an aspect of some embodiments of the present invention there is provided a composite material comprising an effective amount of calcium carbonate-containing material, and an associating moiety being associated with the calcium carbonate-containing material for use in treating a chemical-induced burn or a heat-induced burn in a subject in need thereof.

According to an aspect of some embodiments of the present invention there is provided a method of treating a brittle nail in a subject in need thereof, the method comprising administering to the subject an effective amount of a composite material comprising a calcium carbonate-containing material, and an associating moiety being associated with the calcium carbonate-containing material, thereby treating the brittle nail in the subject.

According to an aspect of some embodiments of the present invention there is provided a composite material comprising an effective amount of calcium carbonate-containing material, and an associating moiety being associated with the calcium carbonate-containing material for use in treating a brittle nail in a subject in need thereof.

According to an aspect of some embodiments of the present invention there is provided a method of treating muscle spasm or tendon inflammation in a subject in need thereof, the method comprising administering to the subject an effective amount of a composite material comprising a calcium carbonate-containing material, and an associating moiety being associated with the calcium carbonate-containing material, thereby treating the muscle spasm or tendon inflammation in the subject.

According to an aspect of some embodiments of the present invention there is provided a composite material comprising an effective amount of calcium carbonate-containing material, and an associating moiety being associated with the calcium carbonate-containing material for use in treating muscle spasm or tendon inflammation in a subject in need thereof.

According to an aspect of some embodiments of the present invention there is provided a method of treating gum inflammation in a subject in need thereof, the method comprising administering to the subject an effective amount of a composite material comprising a calcium carbonate-containing material, and an associating moiety being associated with the calcium carbonate-containing material, thereby treating the gum inflammation in the subject.

According to an aspect of some embodiments of the present invention there is provided a composite material comprising an effective amount of calcium carbonate-containing material, and an associating moiety being associated with the calcium carbonate-containing material for use in treating gum inflammation in a subject in need thereof.

According to an aspect of some embodiments of the present invention there is provided a method of treating glioblastoma in a subject in need thereof, the method comprising administering to the subject an effective amount of a composite material comprising a calcium carbonate-containing material, and an associating moiety being associated with the calcium carbonate-containing material, thereby treating the glioblastoma in the subject.

According to an aspect of some embodiments of the present invention there is provided a composite material comprising an effective amount of calcium carbonate-containing material, and an associating moiety being associated with the calcium carbonate-containing material for use in treating glioblastoma in a subject in need thereof.

According to an aspect of some embodiments of the present invention there is provided a method of treating a bacterially contaminated wound in a subject in need thereof, the method comprising administering to the subject an effective amount of a composite material comprising a calcium carbonate-containing material, and an associating moiety being associated with the calcium carbonate-containing material, thereby treating the bacterially contaminated wound in the subject.

According to an aspect of some embodiments of the present invention there is provided a composite material comprising an effective amount of calcium carbonate-containing material, and an associating moiety being associated with the calcium carbonate-containing material for use in treating a bacterially contaminated wound in a subject in need thereof.

According to some embodiments of the invention, the subject is a human subject.

According to some embodiments of the invention, the subject is a non-human subject.

According to some embodiments of the invention, the administering comprises topical administration or wherein the composite of formulated for topical administration.

According to some embodiments of the invention, the effective amount comprises multiple administrations.

According to some embodiments of the invention, the multiple administrations are affected over a period of at least 7 days.

According to some embodiments of the invention, the composite is formulated in a form of a powder, a gel, a spray, a foam, a mousse, an ointment, a paste, a lotion, a gauze, a wound dressing, a suspension, an adhesive bandage, a non-adhesive bandage, a wipe, a gauze, a pad, an enema, a suppository and a sponge.

According to some embodiments of the invention, the composite further comprises a citrate in chemical association with the calcium carbonate, and/or with the association moiety.

According to some embodiments of the invention, the associating moiety is a positively-charged moiety at physiological pH.

According to some embodiments of the invention, the association moiety is a polymeric moiety.

According to some embodiments of the invention, the association moiety has a molecular weight in a range of 10 to 100 kDa.

According to some embodiments of the invention, the association moiety has a molecular weight of at least 300 kDa.

According to some embodiments of the invention, the association moiety is a biocompatible moiety.

According to some embodiments of the invention, the association moiety is a polypeptide.

According to some embodiments of the invention, the polypeptide comprises at least one amino acid residue that is positively charged at physiological pH.

According to some embodiments of the invention, the polypeptide essentially consists of amino acid residues that are positively charged at physiological pH.

According to some embodiments of the invention, the polypeptide is or comprises a polylysine.

According to some embodiments of the invention, the polylysine is selected poly-D-lysine, poly-L-lysine and poly-ε-lysine.

According to some embodiments of the invention, the polypeptide is or comprises collagen.

According to some embodiments of the invention, the association moiety is or comprises lysine.

According to some embodiments of the invention, the lysine is selected from L-lysine and D-lysine.

According to some embodiments of the invention, the calcium carbonate-containing material comprises crystalline calcium carbonate.

According to some embodiments of the invention, the calcium carbonate-containing material comprises a coral exoskeleton.

According to some embodiments of the invention, the calcium carbonate-containing material comprises amorphous calcium carbonate (ACC) or calcite.

According to some embodiments of the invention, the calcium carbonate-containing material is a particulate material.

According to some embodiments of the invention, the particulate material comprises particles having an average particle diameter in the range of from 0.01 micron to 10 millimeter, or from 0.01 micron to 1 millimeter, or from 0.01 micron to 500 microns, or from 0.1 microns to 500 microns, or from 1 micron to 500 microns, or from 5.0 microns to 500 microns.

According to some embodiments of the invention, the particulate material comprises particles having an average particle diameter in the range of from 0.01 micron to 100 microns, or from 0.01 microns to 50 microns.

According to some embodiments of the invention, the particulate material comprises particles having an average diameter in the range of from 20 nanometer to 10 millimeter, or from 100 nanometer to 1 millimeter.

According to some embodiments of the invention, a weight ratio of the citrate and the calcium carbonate-containing material ranges from 10:1 to 1:10, or from 5:1 to 1:5.

According to some embodiments of the invention, a weight ratio of the association moiety and the calcium carbonate-containing material ranges from 5000:1 to 250:1.

According to some embodiments of the invention, the method further comprises a swelling polymeric moiety.

According to some embodiments of the invention, said swelling polymeric moiety is selected from alginate, chitosan, collagen and a poly(alkylene glycol).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C: A) aphtha ulcer on a tongue. B) treatment with the calcium carbonate, lysine composite (CLC) powder. C) aphtha ulcer one day after treatment.

FIG. 2 shows on the right and left a CLC powder aphtha clamp and a CLC powder aphtha clamp side view.

FIGS. 3A-B: A) Chemotherapy leg's ulcer-contain discontinue of the tissue, skin redness, and inflammation. B) A week post-treatment.

FIGS. 4A-D: A) CLC powder in proctoscope B1) CLC powder embedded in an applicator B2) CLC powder released from the conductor CY CLC powder embedded in glycerin Suppository D) CLC powder embedded in Proctoglivenol Suppository.

FIGS. 5A-D: Injured finger from a knife cut a) at the injury day b) 5 days post-injury c) 11 days post-injury d) 30 days post-injury.

FIGS. 6A-E: Heat injury (burns) located on the anterolateral part of the knee. a) The wound before treatment (2 days post injury) b) treatment with CLC powder (3 days post-injury), the powder covers all the affected area c) bandaging d) 1 day after treatment e) 13 days post treatment.

FIGS. 7A-B: Jellyfish sting injury to the left hand a) two days post injury, before treatment b) one day post treatment.

FIGS. 8A-C: Weak nails and brittle nails a) untreated nail b) CLC-treated nail, 2 days post-treatment c) CLC-treated nail, 14 days post-treatment.

FIGS. 9A-B: Mechanical hit injury located on a finger near the joint. a) The treated wound with CLC ointment 8 minutes post-injury. The upper arrow marks the injury site and the other one the CLC-ointments b) 30 minutes post injury.

FIGS. 10A-C: wounds between the toes, that were caused by itching. a) The wound before treatment b) treatment with CLC powder, the powder covers all the itching area c) A day post treatment.

FIGS. 11A-C: Snaps from control and treated cultures: PI-FDA staining and phase contrast images of A-172 cells after 5 days of treatment with E-CPC. FDA (green) stains the live cells. PI (red) stains the dead cells. Hoechst (blue) stains both live and dead cells. b) Percentage of cell death (PI staining). c) Percentage of total cells (Hoechst staining).

FIGS. 12A-H show a dose-dependent effect of CLC on A172 cells survival. Shown are Hoechst images of total cell nuclei in treated (3 and 5 days) and untreated A172 cells. A-F) Untreated control and treated cells. G-H) % of cell number reduction from control={(total cells in control-total cells in treated)/total cells in control}*100. Error bars indicate the mean with associated S.D. (n=3, ordinary One-way ANOVA, p value 0.033 (*), 0.002 (**), <0.001 (***)).

FIGS. 13A-D show that CLC attach to A172 cells. A) CLC treated cells; B) A magnified image. Arrows point on random CLC particles; C) A CLC particle (arrow) attached to a cell body; D) A CLC particle (arrow) attached to a cell process.

FIGS. 14A-H show morphological changes of CLC treated A172 cells. The morphological changes of treated cells are shown by FDA staining. A, C, E, G) Untreated control cells; B, D, F, H) CLC treated A172 cells. The red arrows mark the cell processes of control and treated cells respectively.

FIGS. 15A-B show the results of an MTT test from which it is evident that CLC reduces the metabolism of A172 cells. Shown are MTT plates and subsequent quantification of the results. A) Colorimetric differentiation of treated cells with respect to control cells. B-C) The effect of treatments on % of inhibition was calculated as {(OD of control-OD of treated)/OD of control}*100. Error bars indicate the mean with associated S.D. (n=3, ordinary One-way ANOVA, p value 0.033 (*), 0.002 (**), <0.001 (***)).

FIGS. 16A-E show that CLC is non-cytotoxic to primary healthy astrocytes. Shown are DAPI images (blue) and primary astrocytes (GFAP, red), at day 19 in culture. The treatment lasted 5 days. A-B) Cell nuclei, control (A) v/s treated (B); C-D) GFAP immunolabeled primary astrocytes, control (C) v/s treated (D). Error bars indicate the mean with associated S.D. (n=3, ordinary One-way ANOVA, p value ns (>0.05)).

FIGS. 17A-E show an analysis of total bacteria using Hoechst staining for Bacterial chromatin. Analysis of percentage of bacterial death using Propidium Iodide for dead bacteria. Number of total live bacteria after subtracting dead bacteria. Two wounds performed on the mice back, the left one showing an untreated wound, while the right one showing a CLC-treated wound. Petri dishes seeded with a swab taken from the wounds 24 hours post-procedure (left-untreated, right-CLC-treated).

### DESCRIPTION OF SPECIFIC EMBODIMENTS OF THE INVENTION

The present invention, in some embodiments thereof, relates to methods of treating.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst conceiving embodiments of the invention, the present inventors realized that a composite material comprising calcium carbonate, an association moiety and optionally citrate can be used to effectively treat various medical conditions of a variety of tissues including skin, deep tissues thereof and brain.

The effectivity of this composite material was demonstrated on a multiple of indications on human beings. As shown in the Examples section which follows, the composite, formulated as powder, gel or ointment treatment was very effective as it immediately, e.g., within seconds/minutes relieved pain, reduced redness, accelerated wound healing process and shortened the healing time of aphthae ulcers, chemotherapy wounds, hemorrhoids and anal fissures, skin injuries-chemical or heat-induced burns, acne, brittle nails, muscle spasm/tendon inflammation (back, knee, neck), dental-gum infection, dental extraction and increased death of glioblastoma brain cancer cells.

The following provides a list of indications contemplated according to some embodiments of the invention.

### Aphtha

Thus, according to an aspect of the invention there is provided a method of treating an aphtha in a subject in need thereof, the method comprising administering to the subject an effective amount of a composite material comprising a calcium carbonate-containing material, and an associating moiety being associated with said calcium carbonate-containing material, thereby treating aphtha in the subject.

Alternatively or additionally, there is a composite material comprising an effective amount of a calcium carbonate-containing material, and an associating moiety being associated with said calcium carbonate-containing material for use in treating aphtha in a subject in need thereof.

Aphtha, also known as canker sores, is a common oral condition characterized by painful ulcerations that occur on the inside of the mouth or on the tongue. Aphtha can be recurrent or occasional and can be categorized into three types: minor, major, and herpetiform, each of which is contemplated herein and considered as a different embodiment.

Minor aphtha is the most common type and is characterized by small, painful ulcers that heal within a few days. Major aphtha is less common but more severe, characterized by larger, deeper ulcers that may take weeks to heal. Herpetiform aphtha is the least common type, characterized by clusters of small, painful ulcers that can last up to a month.

Risk factors for developing aphtha include nutritional deficiencies, hormonal changes, immune system disorders, and genetic predisposition. Nutritional deficiencies, particularly deficiencies in iron, folate, and vitamin B12, have been linked to aphtha development. Hormonal changes, such as those that occur during menstruation or pregnancy, can also increase the risk of aphtha. Immune system disorders, such as HIV or Behcet's disease, can increase the risk of recurrent aphtha. Genetic aphtha is also contemplated herein.

In some embodiments, the composite can be combined with other ingredients to enhance its efficacy in aphtha treatment. For example, the composite can be combined with a pain reliever such as lidocaine or benzocaine to provide rapid relief from pain. The composite can also be combined with a healing agent such as Aloe Vera or honey to promote treatment of the aphtha lesion.

The composite can be applied in any suitable form, such as a topical cream, gel, or paste. The composite can be applied using any suitable means, such as a cotton swab, a spray, brush, or dropper.

The composite can be applied using a dedicated device.

There are several devices that can be used to apply a medicinal to the tongue, including:

Tongue scraper: A tongue scraper is a tool designed to clean the tongue, but can also be used to apply a medicinal to the tongue. It typically has a long, flat surface with a slightly curved edge that can be used to scrape the tongue and apply the medicinal.

Oral syringe: An oral syringe is a plastic or glass device with a long, narrow tip that can be used to administer liquids to the mouth, including medicinal solutions. The tip of the syringe can be placed on the tongue, and the solution can be slowly injected onto the tongue.

Tongue applicator: A tongue applicator is a tool specifically designed for applying medication to the tongue. It typically has a soft, flexible tip that can be used to gently apply the medication to the tongue. A tongue applicator is generally featured with a small wand with a soft, flexible tip. The tip may be made of materials such as silicone or rubber and may be textured or smooth to aid in the application of medication to the tongue. The handle of the applicator may be made of plastic or metal and may have a non-slip grip to make it easier to hold and maneuver. Some tongue applicators may also come with disposable tips for hygiene purposes.

According to an aspect of the invention, the device comprises a pair of contact pads con.red to engage opposite surfaces of a tongue wherein the contact pads are connected by a connecting member, wherein said connecting member is elastic and being configured to apply force on said contact pads toward each other, wherein said connecting member comprises a first segment, a second segment and an apex between said segment (or, in other words, a U-shaped connecting member). An embodiment of such a device is provided in FIG. 2. The active agent (e.g., the composite can be applied on at least one of the contact pads, dependent on the location of the affected area. This device can be used for any tongue related medical condition which requires treatment with a medicament.

Overall, the device selected can be selected based in preference of the subject, and the severity of the condition being treated. It is important to use the device as directed to ensure the proper administration of the medication.

Treatment may be combined with other treatment modalities.

### Chemotherapy-Induced Wounds

According to another aspect of the invention there is provided a method of treating a chemotherapy-induced wound in a subject in need thereof, the method comprising administering to the subject an effective amount of a composite material comprising a calcium carbonate-containing material, and an associating moiety being associated with said calcium carbonate-containing material, thereby treating the chemotherapy-induced wound in the subject.

Alternatively or additionally, there is provided a composite material comprising an effective amount of calcium carbonate-containing material, and an associating moiety being associated with said calcium carbonate-containing material for use in treating a chemotherapy-induced wound in a subject in need thereof.

Various types of cancer are nowadays a serious medical and social problem and a great challenge for modern medicine. The majority of anticancer therapy is based on traditional chemotherapy and radiotherapy. Both of these highly non-specific types of treatment have a number of serious side effects including ulcer formation and wound healing complications. Typically, such wounds are non-bleeding wounds.

Chemotherapy induced wounds may present as ulcers or other types of non-healing wounds.

Some chemotherapy drugs can damage healthy cells in the mouth and cause mouth sores, such as ulcers or infections. Chemotherapy treatment may also reduce the amount of saliva (spit) in your mouth, make your saliva thick or sticky, or make your mouth dry.

According to some embodiments, the wound is a mucosal tissue wound.

According to other embodiments, the wound is in the breast, abdomen, back or at an extremity such as a limb, e.g., legs, palms.

According to a specific embodiment, the wound is a chemotherapy-induced acral erythema. The symptoms can occur anywhere between days to months after administration of the offending medication, depending on the dose and speed of administration. The patient first experiences tingling and/or numbness of the palms and soles. This is followed 2-4 days later by bright redness, which is symmetrical and sharply defined.

In severe cases this may be followed by burning pain and swelling, blistering and ulceration, peeling of the skin.

Types of chemotherapy that causes wounds are typically those belonging to the groups of platinum-based chemotherapy, anthracycline-based chemotherapy, or taxane-based chemotherapy, topoisomerase inhibitors-based chemotherapy, antimetabolites-based chemotherapy, alkylating agents-based chemotherapy.

According to some embodiments, the offending agent that causes chemotherapy-induced acral erythema cabozantinib, cytarabine, doxorubicin, or fluorouracil or its prodrug capecitabine. Targeted cancer therapies, especially the tyrosine kinase inhibitors sorafenib and sunitinib, have also been associated with a high incidence of such wounds and acral erythema is particular.

Treatment may be combined with other treatment modalities.

Modes of administration are provided herein and relate to any form or formulation of topical administration.

### Acne

According to an aspect of the invention there is provided a method of treating acne in a subject in need thereof, the method comprising administering to the subject an effective amount of a composite material comprising a calcium carbonate-containing material, and an associating moiety being associated with said calcium carbonate-containing material, thereby treating the acne in the subject.

Alternatively, or additionally, there is provided a composite material comprising an effective amount of calcium carbonate-containing material, and an associating moiety being associated with said calcium carbonate-containing material for use in treating acne in a subject in need thereof.

As used herein, the term “acne” refers to a skin condition that occurs when hair follicles become plugged with oil and dead skin cells. It is characterized by whiteheads, blackheads pimples, nodules, cysts, oily skin and scarring that can appear on various body structures including but not limited to the face, forehead, chest, upper back and shoulders. The major pathophysiological features of acne include but are not limited to hyperkeratinization, sebum production, bacterial proliferation and inflammation. In some embodiments, acne is associated with inflammatory activity of one or more strains of P. acnes. In some embodiments, acne is characterized by noninflammatory, open or closed comedones and by inflammatory papules, pustules, and nodules. As used herein, acne refers to acne vulgaris, acne conglobata, acne fulminans, Hidradentis suppurativa, scalp acne, acne associated with Progressive Macular Hypomelanosis, acne associated with SAPHO syndrome or acne associated with Fatal Bacterial Granuloma after Trauma. The acne can be any of the following: comedonal acne, cystic acne, nodular acne, acne rosacea, acne mechanice, acne fulimans.

Risk factors for the development of acne, other than genetics, have not been conclusively identified. Possible secondary contributors include hormones, infections, diet, and stress.

Modes of administration are provided herein and relate to any form or formulation of topical administration.

Treatment may be combined with other treatment modalities (e.g., topical retinoids, topical antibiotics, hormonal therapy, benzoyl peroxide, oral antipiotics, Iisotretinoin).

### Hemorrhoids

According to an aspect of the invention there is provided a method of treating hemorrhoids in a subject in need thereof, the method comprising administering to the subject an effective amount of a composite material comprising a calcium carbonate-containing material, and an associating moiety being associated with said calcium carbonate-containing material, thereby treating hemorrhoids in the subject.

Alternatively, or additionally, there is provided a composite material comprising an effective amount of calcium carbonate-containing material, and an associating moiety being associated with said calcium carbonate-containing material for use in treating hemorrhoids in a subject in need thereof.

As used herein “hemorrhoids also called piles, refer to swollen veins in the anus and lower rectum, similar to varicose veins. Hemorrhoids can develop inside the rectum (internal hemorrhoids) or under the skin around the anus (external hemorrhoids). Any stage of hemorrhoids is contemplated herein: first, second, third or fourth.

The method of the invention can be used to effectively treat hemorrhoids of different stages by providing relief from symptoms and pain, reducing inflammation and swelling, and preventing further irritation.

The treatment may be combined with other treatment modalities, such as topical creams or ointments, suppositories, sitz baths, dietary changes (e.g., high fiber diet), surgical intervention, and non-invasive procedures.

Modes of administration are provided herein and relate to any form or formulation of topical administration. The use of enema or a suppository is also contemplated.

### Anal Fissures (Fissures)

An anal fissure is a break or tear in the skin of the anal canal. Anal fissures may be noticed by bright red anal bleeding on toilet paper and undergarments, or sometimes in the toilet. If acute they are painful after defecation, but with chronic fissures, pain intensity often reduces. Anal fissures usually extend from the anal opening and are usually located posteriorly in the midline, probably because of the relatively unsupported nature and poor perfusion of the anal wall in that location. Fissure depth may be superficial or sometimes down to the underlying sphincter muscle. Untreated fissures develop a hood-like skin tag (sentinel piles) which cover the fissure and cause discomfort and pain. Most anal fissures are caused by stretching of the anal mucous membrane beyond its capability.

According to a specific embodiment, the fissure is chronic.

According to other specific embodiments, the fissure is acute.

Treatment may be combined with topical nitroglycerin or calcium channel blockers (e.g. diltiazem), or injection of botulinum toxin into the anal sphincter. Other measures include warm sitz baths, topical anesthetics, high-fiber diet and stool softeners.

Modes of administration are provided herein and relate to any form or formulation of topical administration. The use of enema or a suppository is also contemplated.

### Itching

According to an aspect of the invention there is provided a method of treating itching in a subject in need thereof, the method comprising administering to the subject an effective amount of a composite material comprising a calcium carbonate-containing material, and an associating moiety being associated with said calcium carbonate-containing material, thereby treating itching in the subject.

Alternatively or additionally, there is provided a composite material comprising an effective amount of calcium carbonate-containing material, and an associating moiety being associated with said calcium carbonate-containing material for use in treating itching in a subject in need thereof.

As used herein “itching” or “itch” refers to an unpleasant sensation that causes the desire to scratch the skin. It can be caused by a variety of factors, including allergies, skin diseases, insect bites, and dry skin, each of which is contemplated herein.

In other embodiments, treatment with the composite may be combined with other active ingredients, such as analgesics or anti-inflammatory agents, to provide additional benefits.

Thus, the composite may be used in combination with other treatments, such as antihistamines, steroids, or moisturizers, to provide additional relief from itching.

Modes of administration are provided herein and relate to any form or formulation of topical administration.

### A Chemical-Induced Burn or a Heat-Induced Burn

According to an aspect of the invention there is provided a method of treating a chemical-induced burn or a heat-induced burn in a subject in need thereof, the method comprising administering to the subject an effective amount of a composite material comprising a calcium carbonate-containing material, and an associating moiety being associated with said calcium carbonate-containing material, thereby treating the chemical-induced burn or a heat-induced burn in the subject.

According to an aspect of the invention there is provided a composite material comprising an effective amount of calcium carbonate-containing material, and an associating moiety being associated with said calcium carbonate-containing material for use in treating a chemical-induced burn or a heat-induced burn in a subject in need thereof.

As used herein “a “chemical burn” refers to the result of an exposure of a live tissue to a corrosive substance (such as a strong acid, base or oxidizer) or a cytotoxic agent (such as mustard gas, lewisite or arsine, biotoxins such as those of a jellyfish). Chemical burns follow standard burn classification and may cause extensive tissue damage. The main types of irritant and/or corrosive products are: acids, bases, oxidizers/reducing agents, solvents, and alkylants. Additionally, chemical burns can be caused by biological toxins (such as anthrax toxin) and by some types of cytotoxic chemical weapons, e.g., vesicants such as mustard gas and Lewisite, or urticants such as phosgene oxime.

According to a specific embodiment, the chemical burn is a result of a biotoxin or venoms, e.g., of jellyfish, which are typically composed of potent proteinaccous porins (cellular membrane pore-forming toxins), neurotoxic peptides, bioactive lipids and other small molecules whilst the tubules contain ancient collagens and chitins. Common symptoms of a jellyfish sting include: a burning, stinging sensation on your skin; a tingling or numbness where the sting occurred; the skin in the area where the jellyfish stung turning red or purple.

Heat burn, also known as a thermal burn or a heat injury, is a type of burn that occurs when the skin or other tissues are exposed to high temperatures, such as fire, hot liquids, or steam. Heat burns can cause damage to the skin, nerves, blood vessels, and other tissues, and can vary in severity depending on the duration and intensity of the exposure. Symptoms of a heat burn may include pain, redness, swelling, blistering, and in severe cases, charring or blackening of the affected area.

In general, heat burns are typically caused by temperatures above 44° C. At this temperature, the skin can be damaged in as little as 20 minutes of exposure. Temperatures above 50° C. can cause severe burns in just a few seconds of exposure. The severity of a burn can also depends on the source of the heat—for example, contact with hot metal or oil may cause more severe burns than contact with hot air or water, due to differences in thermal conductivity.

Treatment may be combined with standard treatment such as cooling, cleaning, antibiotics treatment, skin grafting.

### Muscle Spasm and Tendon Inflammation

According to an aspect of the invention there is provided a method of treating muscle spasm or tendon inflammation in a subject in need thereof, the method comprising administering to the subject an effective amount of a composite material comprising a calcium carbonate-containing material, and an associating moiety being associated with said calcium carbonate-containing material, thereby treating the muscle spasm or tendon inflammation in the subject.

Alternatively or additionally, there is provided a composite material comprising an effective amount of calcium carbonate-containing material, and an associating moiety being associated with said calcium carbonate-containing material for use in treating muscle spasm or tendon inflammation in a subject in need thereof.

As used herein “muscle spasm” refers to a sudden involuntary contraction of a muscle, a group of muscles, or a hollow organ, such as the bladder. Various kinds of involuntary muscle activity may be referred to as a “spasm”. A spasm may be a muscle contraction caused by abnormal nerve stimulation or by abnormal activity of the muscle itself. A spasm may lead to muscle strains or tears in tendons and ligaments if the force of the spasm exceeds the tensile strength of the underlying connective tissue. This can occur with a particularly strong spasm or with weakened connective tissue. A hypertonic muscle spasm is a condition of chronic, excessive muscle tone (i.e., tension in a resting muscle). This is the amount of contraction that remains when a muscle is not working. A true hypertonic spasm is caused by malfunctioning feedback nerves. This is much more serious and is permanent unless treated. In this case, the hypertonic muscle tone is excessive, and the muscles are unable to relax.

A subtype of spasm is colic. This is an episodic pain caused by spasm of smooth muscle in a particular organ (e.g., the bile duct). A characteristic of colic is the sensation of having to move about, and the pain may induce nausea or vomiting. Included in the term: Skeletal muscle spasms: These are involuntary contractions of the muscles that attach to bones and can cause cramping, stiffness, and pain. They can occur due to overuse, dehydration, or electrolyte imbalances. Smooth muscle spasms: These are involuntary contractions of the smooth muscles that line the organs and blood vessels. They can cause a variety of symptoms depending on the affected organ, such as abdominal pain, diarrhea, or difficulty breathing. Vocal cord spasm is also contemplated herein.

The mode of administration and formulation would depend on the affected muscle. Both systemic and local administrations (e.g., topical, ocular) are contemplated herein.

Treatments may be combined with any Gold-standard modalities. Examples include, but are not limited to, nonsteroidal anti-inflammatory drugs (NSAIDs): These medications reduce inflammation and pain in the affected muscles. Topical application of calcium can be used in combination with NSAIDs to provide a synergistic effect. Muscle relaxants: These medications relax the muscles and can be used to relieve muscle spasms. Topical application of calcium can be used in combination with muscle relaxants to enhance the therapeutic effect. Physical therapy: This treatment involves exercises and stretches to improve muscle strength and flexibility. Topical application of calcium can be used in combination with physical therapy to provide immediate relief from muscle spasms and improve the effectiveness of the therapy. Electrolyte replacement therapy: This treatment involves replacing the electrolytes in the body, such as potassium and magnesium.

As used herein “tendon inflammation” also known as tendinitis, is a condition in which a tendon becomes inflamed or irritated, causing pain, swelling, and tenderness in the affected area. Tendons are thick cords that attach muscles to bones and help facilitate movement. They are composed of tough, fibrous connective tissue that can become inflamed when subjected to overuse, injury, or disease. Examples of tendons in which inflammation frequently occurs include, but are not limited to, Achilles tendon, Patellar tendon, Rotator cuff tendons and the Biceps tendon.

Ligaments are similar to tendons in that they are composed of connective tissue, but they differ in their function. Ligaments attach bones to other bones and provide stability to joints. Ligament inflammation, also known as a sprain, can occur when a ligament is stretched or torn due to injury or overuse. Examples of ligaments in which inflammation frequently occurs include, but are not limited to, Anterior cruciate ligament (ACL), Posterior cruciate ligament (PCL), Collateral ligaments and ankle ligaments.

Treatment may be combined with other modalities. Examples include but are not limited to physical therapy, rest, elevation, corticosteroid injections.

The mode of administration and formulation would depend on the affected organ, liganemt or tendon, typically, a topical mode of administration is contemplated.

### Brittle Nails

According to an aspect of the invention there is provided a method of treating a brittle nail in a subject in need thereof, the method comprising administering to the subject an effective amount of a composite material comprising a calcium carbonate-containing material, and an associating moiety being associated with said calcium carbonate-containing material, thereby treating the brittle nail in the subject.

Alternatively, or additionally, there is provided a composite material comprising an effective amount of calcium carbonate-containing material, and an associating moiety being associated with said calcium carbonate-containing material for use in treating a brittle nail in a subject in need thereof.

Brittle nails are a common condition characterized by nails that are weak, thin, and prone to splitting, cracking, or breaking easily. There are several possible causes of brittle nails, including: Nutrient deficiencies: Brittle nails can be a sign of nutrient deficiencies, such as a lack of biotin, vitamin C, or iron. Aging: Nails tend to become weaker and more brittle. This is because the natural production of keratin, a protein that makes up the nails, slows down with age. Overexposure to water: Excessive exposure to water, such as frequent hand washing, dishwashing, or swimming, can weaken nails and make them more prone to breakage. Harsh chemicals: Exposure to harsh chemicals, such as cleaning products, solvents, and nail polish remover, can weaken nails and make them more brittle. Medical conditions: Certain medical conditions, such as anemia, thyroid disorders, and psoriasis, can also cause brittle nails.

Treatment may be combined with other common recommendations for a subject in need thereof, such as balanced nutrition rich in vitamins, minerals, and protein to support nail health, use of gloves, and the like.

The composite can be applied using various topical formulations and can also be included in a nail polish.

### Gum Inflammation

According to an aspect of the invention there is provided a method of treating gum inflammation in a subject in need thereof, the method comprising administering to the subject an effective amount of a composite material comprising a calcium carbonate-containing material, and an associating moiety being associated with said calcium carbonate-containing material, thereby treating the gum inflammation in the subject.

Alternatively or additionally, there is provided a composite material comprising an effective amount of calcium carbonate-containing material, and an associating moiety being associated with said calcium carbonate-containing material for use in treating gum inflammation in a subject in need thereof.

Gum infections, such as gingivitis and periodontitis, can be caused by bacterial buildup on teeth and gums. These infections can cause inflammation, bleeding, and tooth loss if left untreated. Dental extractions, on the other hand, can cause pain, swelling, and infection at the extraction site.

The composite can be used alone or with other active ingredients to alleviate gum inflammation. It can be applied using any topical formulation or a dedicated formulation for dental and gum health.

Dental and gum formulations can vary depending on the specific product and its intended use. Some common ingredients and formulations used in dental products that are contemplated herein are listed infra: toothpaste, mouthwash, whitening products (e.g., peroxide or other bleaching agents) that can be formulated as liquids, peroxide or other bleaching agents for example, Tooth fillings: Tooth fillings are typically made of a composite resin material, Denture adhesives: typically contain a combination of ingredients, such as carboxymethylcellulose, petrolatum, and zinc oxide, that create a sticky, yet flexible, barrier between the dentures and the gums.

### Bacterially Contaminated Wounds

According to an aspect of the invention there is provided a method of treating a bacterially contaminated wound in a subject in need thereof, the method comprising administering to the subject an effective amount of a composite material comprising a calcium carbonate-containing material, and an associating moiety being associated with said calcium carbonate-containing material, thereby treating the bacterially contaminated wound in the subject.

Alternatively, or additionally, there is provided a composite material comprising an effective amount of calcium carbonate-containing material, and an associating moiety being associated with said calcium carbonate-containing material for use in treating a bacterially contaminated wound in a subject in need thereof.

According to a specific embodiment, the bacteria are Gram positive.

According to a specific embodiment, the bacteria are Gram negative.

According to a specific embodiment, the bacteria are aerobic.

According to a specific embodiment, the bacteria are anaerobic.

According to a specific embodiment, the bacteria are facultative anaerobic.

According to a specific embodiment, the bacteria is Enterococcus faecalis, Pseudomonas aeruginosa, Streptococcus pyogenes, Enterobacteriaceae, Acinetobacter baumannii or Escherichia coli.

According to a specific embodiment, the bacteria is Staphylococcus (Staph) aureus.

According to a specific embodiment the wound is a skin wound or mucosal wound (e.g., dental wound).

According to a specific embodiment, the bacteria are of the species Staphylococcus aureus, Corynebacterium species or Propionibacterium acnes.

According to a specific embodiment, the bacteria are Gram-positive or Gram-negative, depending on its cell wall composition. According to a specific embodiment, the bacteria are rod-shaped or spherical in shape, or form clusters or chains.

According to a specific embodiment, the composite is used as an anti-septic material.

The treatment with the composite may be combined with other antibiotics (e.g., topical as well as systemic administration. Antibiotics available for topical therapy include for example, aminoglycosides, sulfonamides, and polypeptide antibiotics, as well as metronidazole, fusidic acid, mupirocin, and rctapamulin), pain relievers, analgesics and more.

### Glioblastoma

According to an aspect of the invention there is provided a method of treating glioblastoma in a subject in need thereof, the method comprising administering to the subject an effective amount of a composite material comprising a calcium carbonate-containing material, and an associating moiety being associated with said calcium carbonate-containing material, thereby treating the glioblastoma in the subject.

Alternatively or additionally, there is provided a composite material comprising an effective amount of calcium carbonate-containing material, and an associating moiety being associated with said calcium carbonate-containing material for use in treating glioblastoma in a subject in need thereof.

As used herein, the term “glioblastoma” (GBM), also called glioblastoma multiforme or “grade IV astrocytoma” according to WHO classification refers to a central nervous system primary tumor derived from glial cells. GBM is one of the deadliest human cancers with an incidence of about 3.5/100,000 per year worldwide (Cloughesy, T. F., W. K. Cavence, and P. S. Mischel, Glioblastoma: from molecular pathology to targeted treatment. Annu Rev Pathol, 2014. 9: p. 1-25). Despite the aggressive standard of care currently used including surgery, chemo- and radiotherapy, the prognosis remains very poor with about 15 months overall survival.

According to a particular embodiment, the glioblastoma is at an early stage (e.g. when the tumor is of a diameter of less than 14 mm).

The treatment can be combined with other modalities. The selected treatment regimen can be an aggressive one which is expected to result in the best clinical outcome (e.g., complete cure of the pathology) or a more moderate one which may relief symptoms of the pathology yet results in incomplete cure of the glioblastoma. The type of the adjunct treatment can include a surgical intervention (e.g., removal of lesion, diseased cells, tissue), a chemotherapy (e.g., tubulin inhibitors, such as exatecan, belotecan, Emtansine, etc), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

Thus, the composite may be administered/co-formulated with an immunomodulatory agent. Examples of immunomodulatory agents include immunomodulatory cytokines, including but not limited to, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1 and anti-PDL1; and immunomodulatory drugs including, but not limited to lenalidomide (Revlimid).

As used herein, the term “subject” refers to a human or non-human subject (i.e., veterinary use). General livestock or domesticated animals are contemplated herewith in a specific embodiment, such as mammals or non-mammals, including but not limited to a bird, a cow, a horse, a goat, a sheep, a pig, a dog, a cat, a chicken and a turkey. According to a specific embodiment, the subject in a human being at any age who suffer from a pathology, as listed above, that requires induction of healing or alleviation of inflammatory pain or is at risk for such a medical condition (e.g., brittle nails).

The term “treating” refers to inhibiting, preventing or arresting the development of a pathology (e.g., as listed above) and/or causing the reduction, remission, or regression of a pathology (e.g., as listed above). Those of skill in the art will understand that various methodologies and assays can be used to assess the development of a pathology, and similarly, various methodologies and assays may be used to assess the reduction, remission or regression of a pathology.

As used herein, the term “preventing” refers to keeping condition from occurring in a subject who may be at risk for the condition, but has not yet been diagnosed as having the condition. For example, preventing bacterial contamination of wounds. See above.

### The Composite Material:

As mentioned, treatment according to embodiments of the invention is effected using a composite material, which comprises a calcium carbonate-containing material, a lysine (or poly-D- or poly-L- or poly-epsilon-lysine) being associated with the calcium carbonate-containing material and optionally citrate associated with the lysine (or poly-D- or poly-L- or poly-epsilon-lysine).

By “associated with” it is meant physical and/or chemical association between the indicated components.

Thus, for example, the citrate and/or lysine (or poly-D- or poly-L- or poly-epsilon-lysine) can be attached to the calcium carbonate-containing material, by interacting with the carbonate groups and/or the calcium cations via, e.g., covalent bonds, electrostatic interactions, hydrogen bonding, van der Waals interactions, donor-acceptor interactions, and/or cation-π interactions. These interactions lead to the chemical association between the components.

As an example, and without being bound by any particular theory, the citrate ions may be in chemical association with the positively charged calcium of the calcium carbonate, and/or with positively charged groups of the lysine (or poly-D- or poly-L- or poly-epsilon-lysine). The carbonate groups of the calcium carbonate can also be in chemical association with positively charged groups of the association moiety, while the calcium can be in chemical association with negatively charged group of the association moiety.

Alternatively, the components can be attached to one another by physical association such as surface adsorption, encapsulation, entrapment, entanglement and the likes.

### The Calcium-Carbonate Containing Material:

Herein throughout, the phrase “calcium carbonate-containing material” describes a material, a substance or a composition-of-matter, which is substantially consisting of calcium carbonate, that it, which includes at least 50%, or at least 60%, or at least 70%, or at least 80%, or at least 90%, or at least 95%, or about 100%, by weight, calcium carbonate.

The term “calcium carbonate” as used herein, refers to the chemical compound CaCO₃. In some embodiments, the calcium carbonate is solid calcium carbonate, which can be in crystalline or amorphous form. As used herein, crystalline forms of calcium carbonate include aragonite, calcite, ikaite, vaterite and monohydrocalcite, all of which are encompassed. Other solid forms of calcium carbonate include amorphous calcium carbonate (ACC) or calcite.

Calcium carbonate-containing material usable in the context of the present embodiments can be obtained or derived from natural sources (e.g., from living species or geological matter), or be synthetic (chemically synthesized). Commercially available forms of calcium carbonate are also encompassed.

Natural sources of calcium carbonate include, but are not limited to, rock formations, such as limestone, chalk, marble, travertine and tufa, as well as other geological matters. Calcium carbonate is also a principle structural component of many life forms, and thus can be obtained from, inter alia, corals, plankton, coralline algae, sponges, brachiopods, echinoderms, bryozoa, mollusks and other calcium carbonate-containing organisms.

In some of any of the embodiments described herein, the calcium carbonate-containing material comprises one or more forms of crystalline calcium carbonate.

In some of any of the embodiments described herein, the calcium carbonate-containing material comprises, or consists of, one or more forms of aragonite, calcite, ikaite, vaterite, and monohydrocalcite.

In some of any of the embodiments described herein, the calcium carbonate-containing material comprises aragonite. As used herein, the term “aragonite” refers to the crystalline form of calcium carbonate, which can be commonly found in as mineral deposits in caves and in oceans, and in the shells of mollusks and exoskeleton of cold and warm-water corals. The aragonite can be a geological aragonite or a biogenic aragonite (derived from living species such as corals or mollusks). Geological aragonite typically has a crystalline structure that is different from biogenic aragonite.

In some of any of the embodiments described herein, the calcium carbonate-containing material comprises calcite. As used herein, the term “calcite” refers to a crystalline form of calcium carbonate, differing from aragonite in its crystal lattice form, which can be obtained from sedimentary rocks and from the shells of some marine organisms.

In some of any of the embodiments described herein, the calcium carbonate-containing material comprises both aragonite and calcite.

In some of any of the embodiments described herein, the calcium carbonate-containing material (e.g., aragonite) comprises a coral exoskeleton. The term “coral exoskeleton”, as used herein, refers to the exoskeleton of marine madreporic corals or material derived therefrom. Natural coral (e.g., Porites) consists of a mineral phase, principally calcium carbonate, typically in the structural form of aragonite or calcite, with impurities, such as Sr, Mg and F ions, and an organic matrix. Thus, as used herein, “coral exoskeleton” includes calcium carbonate, e.g., in the form of aragonite or calcite, with or without additional components (minerals, organic and inorganic components) derived from or secreted by the living coral or life forms associated therewith.

The term “coral exoskeleton” is also referred to herein simply as “coral skeleton” or abbreviated as “CS”.

In some of any of the embodiments described herein, the calcium carbonate-containing material is derived from a coral and comprises a coral exoskeleton.

Coral exoskeleton can be a commercially available material (e.g., Biocoral™) and has been reported to be biocompatible and resorbable. Coral-derived material described as coralline HA prepared by hydrothermally converting the original calcium carbonate of the coral Porites in the presence of ammonium phosphate, maintaining the original interconnected macroporosity of the coral, is also commercially-available (Pro Osteon®, Interpore Cross). The high content calcium carbonate coral exoskeleton has been shown to be biocompatible and biodegradable at variable rates depending on porosity, the implantation site and the species.

In some of any of the embodiments described herein, the coral exoskeleton or materials comprising the same are derived from a coral. In some embodiments, the coral can comprise any species, including, but not limited to, Porites, Stylophora, Acropora, Millepora, or a combination thereof.

In some embodiments, the coral is from the Porites species. In some embodiments, the coral is Porites Lutea.

In some embodiments, the coral is from the Acropora species. In some embodiments, the coral is Acropora grandis (which in one embodiment is very common, fast growing, and easy to culture). Acropora samples can be easily collected in sheltered areas of the coral reefs and/or can conveniently be cultured.

In some embodiments, the coral is from the Millepora species. In one embodiment, the coral is Millepora dichotoma. In one embodiment, the coral has a pore size of 150 microns and can be cloned and cultured, making Millerpora useful in the compositions and methods of this invention.

In some embodiments, the coral is from the Stylophora species. Stylophora is a genus of colonial stony corals in the family Pocilloporidae, commonly known as cat's paw corals or birdsnest corals. In some embodiments, the coral is Stylophora subseriata.

In another embodiment, the coral can be from any one or more of the following species: Favites halicora; Goniastrea retiformis; Acanthastrea echinata; Acanthastrea hemprichi; Acanthastrea ishigakiensis; Acropora aspera; Acropora austera; Acropora sp. “brown digitate”; Acropora carduus; Acropora cerealis; Acropora chesterfieldensis; Acropora clathrata; Acropora cophodactyla; Acropora sp. “danai-like”; Acropora divaricata; Acropora donei; Acropora echinata; Acropora efflorescens; Acropora gemmifera; Acropora globiceps; Acropora granulosa; Acropora cf hemprichi; Acropora kosurini; Acropora cf loisettae; Acropora longicyathus; Acropora loripes; Acropora cf lutkeni; Acropora paniculata; Acropora proximalis; Acropora rudis; Acropora selago; Acropora solitaryensis; Acropora cf spicifera as per Veron; Acropora cf spicifera as per Wallace; Acropora tenuis; Acropora valenciennesi; Acropora vaughani; Acropora vermiculata; Astreopora gracilis; Astreopora myriophthalma; Astreopora randalli; Astreopora suggesta; Australomussa rowleyensis; Coscinaraea collumna; Coscinaraea crassa; Cynarina lacrymalis; Distichopora violacea; Echinophyllia echinata; Echinophyllia cf echinoporoides; Echinopora gemmacea; Echinopora hirsutissima; Euphyllia ancora; Euphyllia divisa; Euphyllia yaeyamensis; Favia rotundata; Favia truncatus; Favites acuticollis; Favities pentagona; Fungia granulosa; Fungia klunzingeri; Fungia mollucensis; Galaxea acrhelia; Goniastrea edwardsi; Goniastea minuta; Hydnophora pilosa; Leptoseris explanata; Leptoseris incrustans; Leptoseris mycetoseroides; Leptoseris scabra; Leptoseris yabei; Lithophyllon undulatum; Lobophyllia hemprichii; Merulina scabricula; Millepora dichotoma; Millepora exaesa; Millipora intricata; Millepora murrayensis; Millipore platyphylla; Monastrea curta; Monastrea colemani; Montipora caliculata; Montipora capitata; Montipora foveolata; Montipora meandrina; Montipora tuberculosa; Montipora cf vietnamensis; Oulophyllia laevis; Oxypora crassispinosa; Oxypora lacera; Pavona bipartita; Pavona venosa; Pectinia alcicornis; Pectinia paeonea; Platygyra acuta; Platygyra pini; Platygyra sp “green”; Platygyra verweyi; Podabacia cf lanakensis; Porites annae; Porites cylindrica; Porites evermanni; Porites monticulosa; Psammocora digitata; Psammocora explanulata; Psammocora haimeana; Psammocora superficialis; Sandalolitha dentata; Seriatopora caliendrum; Stylocoeniella armata; Stylocoeniella guentheri; Stylaster sp.; Tubipora musica; Turbinaria stellulata; Stylophora contorta; Stylophora danae; Stylophora kuehlmanni; Stylophora madagascarensis; Stylophora mamillata; Stylophora pistillata; Stylophora subseriata; Stylophora wellsi, or any coral known in the art, or a combination thereof.

Coral exoskeleton is typically porous. In some embodiments, the calcium carbonate-containing material comprises coral exoskeleton having an average pore size (e.g., average diameter) in the range of from 1 micron to 1 millimeter. In one embodiment, the average pore size of a coral ranges from 1 to 200 microns. In one embodiment, the average pore size of a coral ranges from 30 to 180 microns. In one embodiment, the average pore size of a coral ranges from 50 to 500 microns. In one embodiment, the average pore size of a coral ranges from 150 to 220 microns. In one embodiment, the average pore size of a coral ranges from 250 to 1000 microns.

For most therapeutic applications, it is desirable that the calcium carbonate-containing material, when derived from natural sources, such as coral, be devoid of any cellular debris or other organisms associated therewith in its natural state. Thus, in some of any of the embodiments described herein, the coral exoskeleton is an acellular coral exoskeleton.

Calcium carbonate-containing material such as, for example, aragonite, may be a commercially-available material or can be prepared from coral or coral fragments, or from coral sand. Briefly, the coral can be prepared as follows: in one embodiment, coral or coral sand is purified from organic residues, washed, bleached, frozen, dried, sterilized and/or a combination thereof prior to use in the compositions and/or methods of the present embodiments.

In some of any of the embodiments described herein, preparation of the aragonite or coral exoskeleton includes contacting solid coral exoskeleton of a desired size and shape with a solution comprising an oxidizing agent and washing and drying the solid material.

In some of any of the embodiments described herein, the oxidizing agent may be any suitable oxidizing agent, which facilitates the removal of organic debris from the coral exoskeleton. In some embodiments, the oxidizing agent is sodium hypochlorite.

According to this aspect, and in some embodiments, the process comprises conducting the contacting under mildly acidic conditions, so as to remove organic residues and provide acellular coral exoskeleton.

The calcium carbonate-containing material according to some embodiments of the present invention can be provided in a variety of forms, shapes and structures, compatible with a desired application. Some suitable forms and shapes include, but are not limited to, layers, blocks, spherical and hollow spherical forms, concentric spheres, rods, sheets, symmetrical and asymmetrical forms, amorphous and other irregular shapes and particles. The calcium carbonate-containing material can be shaped, for example, to fit a cavity or surface of tissue, or to fit an article containing the composition as described in further hereinafter.

In some of any of the embodiments described herein, the calcium carbonate-containing material (according to any of the respective embodiments and any combination thereof) is provided as particulate calcium carbonate-containing material.

In some embodiments, the particulate material comprises particles having an average particle diameter in the range of from 0.01 micron to 10 millimeter, 0.1 micron to 10 millimeter, or from 0.1 micron to 1 millimeter, or from 0.1 micron to 500 microns, or from 0.5 microns to 500 microns, or from 1 micron to 500 microns, or from 5.0 microns to 500 microns, including any subranges and intermediate values therebetween.

In some of any of the embodiments described herein, a calcium carbonate-containing material is produced from coral or coral sand according to a process comprising washing ground solid calcium carbonate (e.g. aragonite), such as coral or naturally occurring coral sand with water to desalinate it, then disinfecting and drying the desalinated coral sand at temperatures of about 80 degrees to about 150 degrees C., preferably 90 degrees to 120 degrees C., cutting larger pieces of coral into small pieces, and grinding the disinfected and dried coral or coral sand into particles of a desirable average size. In some embodiments, grinding is into particles of a size ranging from 5 to 500 microns.

In some embodiments, coral is ground into particles having a particle diameter of in the range of 1-5, 1-20, 1-50, 1-100, 5-10, 10-15, 15-20, 10-50, 10-100, 20-100, 50-100, 80-150, 100-200, 100-350, 150-500, 1-50 and/or 50-200 microns across, including any intermediate values and subranges therebetween. In some of any of these embodiments, coral is ground to particles having an average particle volume in the range of 1-100, 50-500, 250-1000, 500-2500, 1000-5000 and 2500-10,000 cubic micron or 0.01-0.1, 0.05-0.5, 0.5-0.75, 0.75-1.0, 1.0-2.0 and 1.0-5.0 cubic millimeters in volume, including any intermediate values and subranges therebetween.

In some of any of the embodiments described herein, the calcium carbonate-containing particulate material, including ACC and/or CS and/or any other material as described herein, comprises particles having a relatively small average particle diameter, for example, in a range of from 0.01 micron to 100 microns, 0.1 micron to 100 microns or from 0.1 microns to 50 microns, including any intermediate values and subranges therebetween.

In some of any of the embodiments described herein, the calcium carbonate-containing particulate material, including ACC and/or CS and/or any other material as described herein, comprises particles having a relatively large average particle diameter, for example, an average diameter higher than 50 microns, for example, in the range of from 50 microns to 10 millimeter, or from 50 microns to 1 millimeter, or from 100 microns to 1 millimeter.

In some of any of the embodiments described herein, the calcium carbonate-containing particulate material, including ACC and/or CS and/or any other material as described herein, comprises a mixture of particles having a relatively large average particle diameter, as described herein, and particles having a relatively small average particle diameter, as described herein.

Exemplary calcium carbonate-containing materials are described in the Examples section that follows.

### The Citrate:

As used herein, the term “citrate salt” describes a compound composed of a citrate ion and one or more cations. The citrate ion can be represented by the formula C₆H₅O₇³⁻ or C₃H₅O(COO)₃³⁻. The cation can be monovalent, divalent or trivalent cation, and the stoichiometry of the citrate ion is in accordance with the selected cation.

The cation can be Na⁺, K⁺, Li⁺, Mg⁺², Zn⁺², Fe⁺², Fe⁺³, and any other suitable cation. If the cation is a monovalent cation, such as, for example, sodium cation, the citrate salt comprises 3 cations, and is, for example, tri-sodium citrate.

In some of any of the embodiments described herein, other salts of multicarboxylic acids can be used as alternative, or in addition, to a citrate salt as described herein.

By “multicarboxylic acid” it is meant an organic compound featuring two, three or more carboxylic acid groups. For a non-limiting example, a multicarboxylic acid can be represented by R(COOH)n, with R being an alkyl, alkenyl, cycloalkyl, and/or aryl, and n being an integer of at least 2 (e.g., 2, 3, 4, 5, etc.). The alkyl, alkenyl, cycloalkyl, or aryl, can be further substituted by one or more other substituents, as described herein.

In some of any of the embodiments described herein, other calcium-chelating agents can be used as alternative, or in addition, to a citrate salt as described herein.

In some of any of the embodiments described herein, other anti-coagulants can be used as alternative, or in addition, to a citrate salt as described herein. In some embodiments, such anti-coagulants are those acting by effecting the formation of cross-linked fibrin. In some embodiments, such anti-coagulants are not acting by effecting platelet aggregation. In some embodiments, the anti-coagulant is other than heparin or similarly-acting anti-coagulants that effect platelet aggregation.

### The Associating Moiety:

The associating moiety is aimed at associating the calcium carbonate-containing material and the citrate so as to form a composite material.

As discussed hereinabove, and without being bound to any particular theory, it is assumed that the associating moiety is such that can form physical and/or chemical interactions with one or both of the calcium carbonate-containing material and the citrate.

In some embodiments, the associating moiety is such that can form electrostatic interactions with one or more of the calcium carbonate-containing material and/or the citrate.

In some embodiments, the associating moiety features functional groups that are positively charged or negatively charged at physiological pH. Positively charged groups can form electrostatic interactions with the citrate and/or the carbonate, the latter leading to release of calcium ions. Negatively charged groups can for electrostatic interactions with calcium ions.

In some of any of the embodiments described herein, the associating moiety comprises one or more positively charged groups, and may further comprise one or more negatively charged groups.

According to some of any of the embodiments described herein, the association moiety is a biocompatible moiety.

In exemplary embodiments, the associating moiety is a positively-charged moiety at physiological pH.

The associating moiety can be a polymeric moiety or a non-polymeric moiety.

In some embodiments, the associating moiety is a polymeric moiety.

The polymeric moiety can be a large polymeric moiety, having a molecular weight higher than 100 kDa, or higher than 200 kDa, or 300 kDa or higher than 300 kDa, for example, in a range of from about 100 to about 1000, or from about 200 to about 1000, or from about 300 to about 1000, or from about 300 to about 800, or from about 300 to about 600, kDa, including any intermediate values and subranges therebetween.

The polymeric moiety can be a large polymeric moiety, having a molecular weight of 100 kDa or lower, for example, in a range of from about 10 to about 100, or from about 20 to about 100, or from about 30 to about 100, or from about 30 to about 80, or from about 30 to about 60, kDa, including any intermediate values and subranges therebetween.

Whenever a molecular weight or MW is described herein in the context of polymeric moieties (e.g., polypeptides), it is meant an average molecular weight, typically determined by conventional methods known in the art and/or in accordance with an information provided by the vendor thereof.

In some of any of the embodiments described herein, the associating moiety is a polymeric moiety, preferably a biocompatible polymeric moiety. In exemplary embodiments, the associating moiety is a polypeptide, featuring high or low molecular weight as described herein.

The term “polypeptide” as used herein encompasses native peptide macromolecules, including degradation products, synthetically prepared peptides and recombinant peptides (e.g., recombinantly expressed in a microorganism), as well as peptidomimetic macromolecules (typically, synthetically synthesized peptides), as well as peptoid and semipeptoid macromolecules which are peptide analogs, which may have, for example, modifications rendering the polypeptides more stable. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided herein below.

Peptide bonds (—CO—NH—) within the polypeptide may be substituted, for example, by N-methylated amide bonds (—N(CH₃)—CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g. methyl), amine bonds (—CH₂—NH—), sulfide bonds (—CH₂—S—), ethylene bonds (—CH₂—CH₂—), hydroxyethylene bonds (—CH(OH)—CH₂—), thioamide bonds (—CS—NH—), olefinic double bonds (—CH═CH—), fluorinated olefinic double bonds (—CF═CH—), retro-amide bonds (—NH—CO—), peptide derivatives (—N(R)—CH₂—CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some of any of the embodiments described herein may also include one or more modified amino acids or one or more non-amino acid monomers e.g. fatty acids, complex carbohydrates, etc.

The term “amino acid” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The polypeptides of some embodiments of the invention are preferably utilized in a linear form, although it will be appreciated that in cases where cyclization does not severely interfere with polypeptide characteristics, cyclic forms of the polypeptide can also be utilized.

The polypeptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing polypeptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide compound. By simple modification of this general procedure, it is possible to add more than one amino acid at a time to a growing chain, for example, by coupling (under conditions which do not racemize chiral centers) a protected tripeptide with a properly protected dipeptide to form, after de-protection, a pentapeptide and so forth. Further description of polypeptide synthesis is disclosed in U.S. Pat. No. 6,472,505. Large scale polypeptide synthesis is described, for example, by Andersson [Biopolymers 2000; 55 (3): 227-50].

Polypeptides of the present invention can be purified using a variety of standard protein purification techniques, such as, but not limited to, heat treatments, salting out for example with ammonium sulfate, polyethyleneimines (PEI) precipitation, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

According to some of any of the embodiments described herein, the associating moiety is a polypeptide, as described herein, and the polypeptide comprises one or more amino acid residue(s) that is/are positively charged at physiological pH.

Such amino acid residues typically comprise a primary or secondary amine group at the side chain thereof, and include, for example, the naturally occurring L-lysine, L-arginine, and L-histidine, and non-naturally occurring amino acid analogs thereof, such as, for example, D-lysine, D-arginine, D-histidine, &-Lysine and ornithine.

In some of these embodiments, at least 5%, or at least 10%, or at least 20%, or at least 30%, or at least 40%, or at least 50%, or more, or substantially all, of the amino acid residues in the polypeptide are positively charged at physiological pH. The positively charged amino acid residues can be dispersed randomly within the polypeptide, and can include one or more types of positively charged amino acid residues, such as described herein.

In some of these embodiments, the polypeptide is consisted of amino acid residues that are positively charged amino at physiological pH, and can be, for example, polylysine, polyhistidine, polyarginine, polyornithine, etc.

In exemplary embodiments, the polypeptide comprises a plurality (e.g., at least 20%, or at least 50%, or at least 80%, or 100%) of lysine residues, which can be L-lysine residues and/or D-lysine residues. Alternatively, or in addition, the lysine residues are ε-lysine residues.

In exemplary embodiments, the polypeptide comprises a plurality (e.g., at least 20%, or at least 50%, or at least 80%, or 100%) of arginine residues, which can be L-arginine residues and/or D-arginine residues.

In exemplary embodiments, the polypeptide comprises a plurality (e.g., at least 20%, or at least 50%, or at least 80%, or 100%) of histidine residues, which can be L-histidine residues and/or D-histidine residues.

In exemplary embodiments, the polypeptide comprises a plurality (e.g., at least 20%, or at least 50%, or at least 80%, or 100%) of ornithine residues, which can be L-ornithine residues and/or D-ornithine residues.

In exemplary embodiments, the polypeptide is poly-D-lysine (PDL).

In exemplary embodiments, the polypeptide is poly-L-lysine (PDL).

In some embodiments, the polypeptide is &-poly-Lysine (e.g., L-lysine).

Other polypeptides are also contemplated, for example, collagen (e.g., Types I, II and III), preferably human collagen, which can be synthetically prepared, recombinant, or extracted from a natural source. In exemplary embodiments, the collagen has average molecular weight (MW) that ranges from about 100 to about 200 kDa.

Any of the polypeptides described herein can have a low or high molecular weight as described herein.

According to some of any of the embodiments described herein, the associating moiety is a non-polymeric moiety.

The non-polymeric moiety can be, for example, positively charged at physiological pH.

The non-polymeric moiety can be, for example, an amino acid that is positively charged at physiological pH, as described herein in any of the respective embodiments.

In exemplary embodiments, the amino acid is L-lysine and/or D-lysine.

In exemplary embodiments, the amino acid is arginine, histidine, ornithine or ε-lysine.

In some embodiments, the associating moiety is capable of interfering in (e.g., inhibiting) a fibrinolysis process in a subject. Exemplary such associating moieties are positively charged polymeric and/or non-polymeric moieties as described herein (e.g., polypeptides and/or amino acids), and/or moieties that structurally resemble coagulants such as tranexamic acid or aminocaproic acid.

According to exemplary embodiments, the associating moiety is poly-D-lysine, poly-L-lysine, poly-D-L-lysine (with any ratio of the D-lysine and L-lysine), each having a high or low molecular weight as described herein, or any mixture thereof.

According to exemplary embodiments, the associating moiety is D-lysine, L-lysine, or a mixture thereof.

### The Composite Material:

The composite material of the present embodiments comprises the citrate, calcium-carbonate-containing material and the associating moiety, associated to one another, and encompasses any form of association, as described herein, between these components, and at any order.

In an exemplary configuration, at least a portion of the association moiety is deposited onto at least a portion of the surface of the calcium carbonate-containing material. In some of these embodiments, the calcium carbonate-containing material is a particulate material and the association moiety is deposited on a portion of the surface or practically coats the surface of at least a portion or all of the calcium carbonate-containing material particles.

In some of these embodiments, at least a portion of the calcium carbonate-containing material is associated with at least a portion of the association moiety via electrostatic interactions formed between the carbonate of the calcium carbonate-containing material and a positively charged group of the association moiety.

Further in this exemplary configuration, at least a portion of the citrate is associated with the portion of association moiety which is deposited onto a surface of the particulate material. Alternatively, or in addition, the citrate is associated with the calcium carbonate-containing material as described herein.

In some of these embodiments, at least a portion of the citrate is associated with at least a portion of the association moiety via electrostatic interactions and/or hydrogen bond interactions.

In some of any of the embodiments described herein, a weight ratio of the citrate and the calcium carbonate-containing material ranges from 10:1 to 1:10, or from 5:1 to 1:5, including any intermediate values and subranges therebetween, and can be, for example, 10:1, 5:1, 2:1, 1:1, 1:2, 1:5 or 1:10.

In some of any of the embodiments described herein, a weight ratio of the associating moiety and the calcium carbonate-containing material ranges from 5000:1 to 250:1, or from 2500:1 to 250:1, including any intermediate values and subranges therebetween, and can be, for example, 5000:1, 2500:1, 1000:1, 500:1 or 250:1.

The composite material may further comprise one or more additional components which may complement its function, such as antibiotics. Other contemplated ingredients are listed above as exemplary embodiments.

Preferably, the additional components are selected such as they do not interfere with the association and performance of the citrate, the calcium carbonate-containing material and the associating moiety.

According to a specific embodiment, the composite is CLC which stands for calcium carbonate, lysine (as the associating moiety e.g., L, D) and citrate.

When the lysine is epsilon poly-lysine, it is termed “E-CPC”.

In exemplary embodiments, the composite material may further comprise collagen, which is in association with the calcium carbonate, the associating moiety and/or the citrate.

According to an aspect of some embodiments of the present invention there is provided a composite material that comprises a calcium carbonate-containing material as described herein in any of the respective embodiments and any combination thereof, and an associating moiety as described herein in any of the respective embodiments and any combination thereof.

### Compositions:

The composite material described herein can be used per se, or can be formulated together with a pharmaceutically acceptable carrier, to form a composition, e.g., a pharmaceutical composition.

As used herein, the term “pharmaceutically acceptable carrier” describes a carrier or a diluent that is used to facilitate the administration of the composite material (also referred to in this context as an active ingredient or active agent) or of the composition containing same and which does not cause significant irritation to an organism and does not abrogate the biological activity and properties of the administered active materials. Examples, without limitations, of carriers include water, buffered aqueous solutions, propylene glycol, emulsions and mixtures of organic solvents with water, as well as solid (e.g. powdered or polymeric) and gaseous carriers.

Techniques for formulation and administration of drugs may be found in “Remington's Pharmaceutical Sciences” Mack Publishing Co., Easton, PA, latest edition, which is incorporated herein by reference.

Compositions for use in accordance with the present embodiments thus may be formulated in conventional manner using one or more pharmaceutically acceptable carriers, excipients and/or auxiliaries, which facilitate processing of the compounds into preparations which can be used pharmaceutically. The dosage may vary depending upon the dosage form employed and the route of administration utilized.

The exact formulation, route of administration and dosage can be chosen by the individual physician in view of the patient's condition (see e.g., Fingl et al., 1975, in “The Pharmacological Basis of Therapeutics”, Ch. 1 p. 1).

The pharmaceutically acceptable carrier can be either an organic carrier or an aqueous carrier. In some embodiments, the carrier is an aqueous carrier. An aqueous carrier preferably comprises injectable-grade water, i.e., USP grade “water for injection”. However, other forms of purified water may be suitable, such as, for example, distilled and deionized water.

Aqueous formulations are preferred since these formulations are gentle to bodily tissues and are suitable for use on wounds. However, non-aqueous formulations are also contemplated. For example, in cases where the composition is in a form of a paste or an emulsion, non-aqueous carriers or mixed carriers of aqueous and organic carriers can be used.

The composition may be formulated for administration in either one or more of routes, depending on the area to be treated.

According to some embodiments, the composition is formulated for topical application, as a topical dosage form.

As used herein, the phrase “topical dosage form” describes a dosage form suitable for topical administration to the treated area (e.g., wound). By “topical administration” it is meant application onto the treated skin area, or “local administration”.

The compositions described herein can be, for example, in a form of a powder, granules, a cream, an ointment, a paste, a gel, a lotion, a milk, a suspension, an aerosol, a spray, a foam, a gauze, a wipe, a sponge, a wound dressing, a pledget, a patch, a pad, an adhesive bandage, and a non-adhesive bandage.

According to some embodiment, the composite is formulated in the form of a powder, a gel, a spray, a foam, a mousse, an ointment, a paste, a lotion, a gauze, a wound dressing, a suspension, an adhesive bandage, a non-adhesive bandage, a wipe, a gauze, a pad, and a sponge.

In some embodiments, the composition is formulated as a liquid reservoir, to be applied as drops, spray, aerosol, liquid, foam, enema and the like. Suitable carriers and other ingredients are used in these cases. For example, for application as an aerosol or foam, a propellant is used. For application as foam, foam-forming agents can also be used.

In some embodiments, the composition is formulated as a cream. Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information. An exemplary cream formulation can be obtained by mixing the composite material described herein with a carrier comprising cellulose derivatives such as cellulose acetate, hydroxyethyl cellulose and/or a polyethylene glycol.

In some embodiments, the composition is formulated as an ointment. Ointments are semisolid preparations, typically based on petrolatum or petroleum derivatives. The specific ointment base to be used is one that provides for optimum delivery for the active agent chosen for a given formulation. As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

In some embodiments, the composition is formulated as a lotion. Lotions are preparations that are to be applied to the skin surface without friction. Lotions are typically liquid or semiliquid preparations in which solid particles, namely, the calcium carbonate-containing material particles, are present in a water or alcohol base. Lotions are typically preferred for covering/protecting large body areas, due to the case of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

In some embodiments, the composition is formulated as a paste. Pastes are semisolid dosage forms in which the bioactive agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

In some embodiments, the composition is formulated as a gel. Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

In some embodiments, the composition is formulated as a foam. Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropanc. Application and agitation of such a composition at the body temperature cause the isopropane to vaporize and generate the foam, in a manner similar to a pressurized aerosol foaming system. Foams can be water-based or hydroalcoholic, but are typically formulated with high alcohol content which, upon application to the treated area, quickly evaporates, driving the composite material to the site of treatment.

In some embodiments, the composition is formulated as a powder or granules. Such compositions can optionally be prepared by preparing the composite material (e.g., as described herein) and forming granules or beads containing these ingredients, for example, by adding suitable agents (e.g., water soluble film-forming agents).

In some embodiments, a topical dosage form includes a solid or semi-solid substrate, e.g., a gauze, a wipe, a bandage, a pad, a pledget, a sponge, a mesh, a fabric, and the likes, and the composite material is incorporated in and/or on the substrate.

The substrate in such topical dosage forms can be of any form and materials used to make up gauzes, wipes, bandages, pads, pledgets, sponges, meshes, fabrics (woven and non-woven, cotton fabrics, and the like), and any other substrates commonly used in medical applications.

Such topical dosage forms may optionally further comprise an adhesive, for facilitating the topical application of the composition onto the treated area for a prolonged time period.

Exemplary adhesives include, but are not limited to, medically acceptable bioadhesives, polymer glues, etc., and can be applied to the substrate by, for example, dip coating with an adhesive base. Such dip coating can be effected during manufacture of the substrate, or at any time prior to its application. In some embodiments, the composite material can be embedded within and/or on the material of the substrate, for example, embedded into or onto a polymer or fabrics by application of heat, or fused to the substrate. In other embodiments, the composite material can be incorporated into the base material of the substrate, for example, mixed within the components of a polymer before polymerization, or mixed with components forming fibers used to make up a gauze or a mesh or pad, etc.

The composition described herein can further comprise additional ingredients, which are aimed at improving or facilitating its preparation, application and/or performance. Such additional ingredients include, for example, anti-irritants, anti-foaming agents, humectants, deodorants, antiperspirants, preservatives, emulsifiers, occlusive agents, emollients, thickeners, penetration enhancers, colorants, propellants and/or surfactants, depending on the final form of the composition.

Representative examples of humectants that are usable in this context of the present embodiments include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

Representative examples of deodorant agents that are usable in the context of the present embodiments include, without limitation, 2,4,4′-trichloro-2′-hydroxy diphenyl ether, and diaminoalkyl amides such as L-lysine hexadecyl amide.

Suitable preservatives that can be used in the context of the present embodiments include, without limitation, one or more alkanols, parabens such as methylparaben and propylparaben, propylene glycols, sorbates, urea derivatives such as diazolindinyl urea, or any combinations thereof.

Suitable emulsifiers that can be used in the context of the present embodiments include, for example, one or more sorbitans, alkoxylated fatty alcohols, alkylpolyglycosides, soaps, alkyl sulfates, or any combinations thereof.

Suitable occlusive agents that can be used in the context of the present embodiments include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients, that can be used in the context of the present embodiments include, for example, dodecane, squalane, cholesterol, isohexadecane, isononyl isononanoate, PPG ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners that can be used in the context of the present embodiments include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, and mixtures thereof.

Suitable penetration enhancers usable in context of the present embodiments include, but are not limited to, polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, menthol, TWEENS such as TWEEN 20, and the like. The permeation enhancer may also be a vegetable oil. Such oils include, for example, safflower oil, cottonseed oil and corn oil.

Suitable anti-irritants that can be used in the context of the present embodiments include, for example, steroidal and non-steroidal anti-inflammatory agents or other materials such as menthol, aloe vera, chamomile, alpha-bisabolol, cola nitida extract, green tea extract, tea tree oil, licorice extract, allantoin, caffeine or other xanthines, glycyrrhizic acid and its derivatives.

Any of the additional ingredients or agents described herein is preferably selected as being compatible with the components of the composite material as described herein, such that there is no interference with the availability of these materials in the composition.

Any of the additional ingredients described herein is further preferably selected as being biocompatible.

In some embodiments, the composition further comprises an additional therapeutically active agent, for example, an additional hemostatic agent or composition or article, or, for example, an agent capable of disinfecting the treated area (e.g., antiseptic agents or compositions).

It will be appreciated that systemic treatment is also contemplated herewith.

This is of a specific relevance for indications such as glioblastoma.

The composite may be formulated for crossing the blood brain barrier.

Exemplary methods for formulating the above described agents to enhance its penetration across the blood brain barrier are described in Yeini et al., Advanced Therapeutics, DOI: 10.1002/adtp.202000124.

Thus, for example, the agents can be formulated in nanoparticles such as liposome-based nanoparticles, amphiphilic micelles, dendrimers, inorganic nanoparticles and polymeric nanoparticles.

Specifically, for the delivery of oligonucleotides, the use of cationic nanoemulsions modified biodegradable poly(β-Amino Ester) (PBAE), cell derived extracellular vesicles, spherical nucleic acid nanoparticles, may be considered to improve delivery to the brain.

Since the BBB restricts the passage of most therapeutic agents from the blood to the brain, receptor-mediated transcytosis can offer a non-invasive trafficking system to deliver targeted carriers into the brain parenchyma. In addition, this approach allows selective targeting of tumor cells within the brain tissue, thus reducing toxicity in other tissues and non-tumor cells in the brain. Examples of receptor-mediated approaches include manipulation of the apolipoprotein receptor, targeting of the epidermal growth factor receptor, transferrin receptor targeting, insulin receptor targeting and adhesion molecule targeting are all contemplated.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into the brain of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluorocthane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions. Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Dosage amount and interval may be adjusted individually to provide brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is affected or diminution of the disease state is achieved.

According to a particular embodiment, the active agent is administered following resection of said glioblastoma tumor.

The amount of a composition to be administered will, of course, be dependent on the subject being treated, the severity of the affliction, the manner of administration, the judgment of the prescribing physician, etc.

Generally (local or systemic formulationa) compositions of the present embodiments may, if desired, be presented in a pack or dispenser device, such as an FDA (the U.S. Food and Drug Administration) approved kit, which may contain one or more unit dosage forms containing the composite material. The pack may comprise, for example, glass or plastic foil. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug

Administration for prescription drugs or of an approved product insert. Compositions of the invention formulated in a compatible pharmaceutical carrier may also be prepared, placed in an appropriate container, and labeled for a medical indication, as detailed herein.

The compositions described herein may be packed or presented in any convenient way. For example, they may be packed in a tube, a bottle, a dispenser, a squeczable container, or a pressurized container, using techniques well known to those skilled in the art and as set forth in reference works such as Remington's Pharmaceutical Science 15th Ed. It is preferred that the packaging is done in such a way so as to minimize contact of the unused compositions with the environment, in order to minimize contamination of the compositions before and after the container is opened.

The compositions described herein are preferably supplied in the concentration intended for use but may also be prepared as concentrates that are diluted prior to use. For example, concentrates requiring dilution ratios of 2:1 to 10:1 parts carrier to a concentrate are contemplated.

In some embodiments, the composition described herein is packaged in a packaging material and identified in print, in or on the packaging material, for use in treating chronic wounds, nociceptive pain or non-bleeding wounds, as described herein.

Articles-of-manufacturing:

According to an aspect of some embodiments of the present invention, an article-of-manufacturing is provided, which comprises the composite material or the composition as described herein in any of the respective embodiments, and any combination thereof, and means for topically applying the composite material or a composition comprising same onto the treated area. In some embodiments, the article-of-manufacturing is configured to apply the composition to wounds.

In some embodiments, the article-of-manufacturing comprises the composition as described herein, in a form of a suspension, packaged in a container, and means for applying the composition as drops, spray, aerosol, foam, using techniques well known to those skilled in the art and as described herein.

In some embodiments, the article-of-manufacturing comprises the composition as described herein, in a form of a cream, lotion, paste, ointment, and the likes, packaged in a suitable container, and optionally comprising means for dispensing the composition from the container.

In some embodiments, the article-of-manufacturing comprises the composite material or a composition comprising same as described herein, in a form that comprises a powder or granules, packaged in a suitable container, and optionally comprising means for dispensing the composition from the container.

In some embodiments, the article-of-manufacturing comprises the composite material or a composition comprising same as described herein, incorporated in and/or on a substrate, as described herein. The article-of-manufacturing can be packaged in a sterile packaging. In exemplary embodiments, the substrate is a gauze or any other solid substrate usable in medical applications, and the article-of-manufacturing is bandage comprising the composite material.

The article-of-manufacturing can be labeled as described herein, for example, by being identified in print, in or on the packaging material, for use in treating chronic wounds, nociceptive pain or non-bleeding wounds.

### Kits:

According to an additional aspect of embodiments of the invention there is provided a kit, which comprises the composite material or a composition comprising same as described herein, being packaged in a packaging material.

The kit can be labeled, for example, by being identified in print, in or on the packaging material, for use in treating chronic wounds, nociceptive pain or non-bleeding wounds.

The components of the composition can be packaged within the kit either together, as a single, ready for use, composition, or at least one of the components (e.g., a carrier or a solid substrate) can be packaged individually. When one or more components are packaged individually, the kit may further be supplied with instructions indicating the route of to prepare and/or apply the components so as to contact an area to be treated with the composite material or the composition. Such instructions can be, for example, mixing the components (e.g., the composite material and the carrier) prior to application or simultaneously or subsequently applying the components (e.g., the composite material and the carrier) onto an area to be treated.

In an exemplary embodiment, the kit comprises the composite material in a first container (e.g., a sterile packaging), and a liquid carrier packaged individually (e.g., in another container), and instructions to add the carrier to the first container, prior to application of the composition to the area to be treated, or vice versa (instructions to add the content of the first container to the carrier in the second container). The first (or second) container can be configured to apply the composition as drops, spray, aerosol, foam, etc. The kit may alternatively further comprise a device for dispensing the composition.

In another exemplary embodiment, the kit comprises the composite material in a first container (e.g., a sterile packaging), and a solid carrier (e.g., a gauze) packaged individually (e.g., in another container), and instructions to contact the composite material with the solid carrier to provide a composition, prior to application of the composition to the area to be treated or instructions to contact a wounded area to be treated with the composite material and apply the gauze or other solid substrate onto the applied composite material. The kit may alternatively further comprise a device for dispensing the composite material.

In another exemplary embodiment, the kit comprises the calcium carbonate-containing material, the association moiety and the citrate, each packaged individually, and each optionally together with a carrier, or a carrier is optionally packaged individually with the kit. The carrier is such that is suitable for the selected dosage form, as described herein. The kit further comprises instructions to prepare the composite material, as described herein, and optionally mix the composite material with the carrier or otherwise use the composite material in combination with the carrier as described herein.

The containers, substrates, and compositions included in the kit can be in accordance with any of the embodiments described herein, and any combination thereof.

### Treatment Regimen

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. The dosage, schedule and duration of treatment can vary, depending on the severity of pathology and the selected type of treatment, and those of skills in the art are capable of adjusting the type of treatment with the dosage, schedule and duration of treatment.

According to some embodiments, the regimen includes multiple administrations.

Thus, for instance, the composite or composition comprising same ban be administered once a day or a number of times a day (e.g., at least 2, 3, 4) for a plurality of days, e.g., at least 3, 7, 10, 14, 21, 30 days, one month, two months, 3 months, 4 months, 5 months, 6 months, 7 months, 8 months, 9 months, 10 months, 11 months, 12 months, 18 months, 24 months or more. Alternatively, the composite or composition comprising same ban be administered once a day or a number of times a day (e.g., at least 2, 3, 4) for a plurality of days but dependent on the severity. Hence there can be periods, e.g., 2, 3, 7, 10 days in which the subject does not treat with the composite or composition comprising same and treatment may be resumed as needed.

According to a specific embodiment, the multiple administrations are effected over a period of at least 7 days.

It will be appreciated that a single treatment or an acute treatment not exceeding 2, 3 days is also contemplated such as for the treatment of nociceptive pain.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Eliminating Aphthae Ulcers

Aphthae, also known as canker sores, are small, shallow ulcers that occur on the mucous membranes of the mouth, such as the inside of the cheeks, lips, and on or under the tongue. They are usually white or yellow in color, and have a red border. Aphthae are not contagious and are not caused by an infection. The exact cause of aphthae is unknown, but they may be triggered by factors such as stress, certain foods, and hormonal changes. The healing time can vary depending on a number of factors, including the severity of the ulcers and the individual's overall health. On average, aphthae typically heal within 7 to 10 days without treatment. Treatment typically includes pain relievers, such as ibuprofen or acetaminophen and topical creams or gels, such as those containing benzocaine or hydrocortisone.

In more severe cases, a prescription medication, such as a steroid ointment or a mouthwash containing an antiseptic and a mild anesthetic, is recommended. Some individuals experience discomfort or pain when applying topical creams or gels to the ulcers.

### Materials and Methods

### CLC Preparation:

The following process was performed in sterile conditions. Calcium Carbonate (Glentham, GK3384, CAS: 471-34-1) powder was transferred into a glass beaker and sterilized in an autoclave. The powder was then transferred into a 0.2 mg/ml L-Lysine solution (Sigma-Aldrich, L5501, CAS: 56-87-1) (powder: liquid ratio 1 gr: 2 ml) and incubated overnight (preferable at 4° C.) under stirring. The next day, the mixture was centrifuged at a speed of 1000 g in order to separate it from the liquid and then resuspended in double distilled water (DDW) to perform two washes (using centrifugation between them to replace the liquid). After washing, the powder was suspended in a 3.2% Sodium Citrate solution (powder: liquid ratio 1 gr: 2 ml) (Merck, 1.06448. CAS: 6132-04-03) for 5 minutes at room temperature. Two additional washes were performed with DDW and the powder was then transferred to an aluminum mold for airdrying in a biological hood. After complete drying, the powder was transferred into test tubes and kept until use at 4° C.

### Treatment

Four subjects (3 adults, age 30-63 and 1 child, age 12), with minor aphthae ulcers in their mouth/tongue were treated with 2 mg CLC. The subjects covered the wounds with the powder using an applicator for 15-30 minutes.

### Results

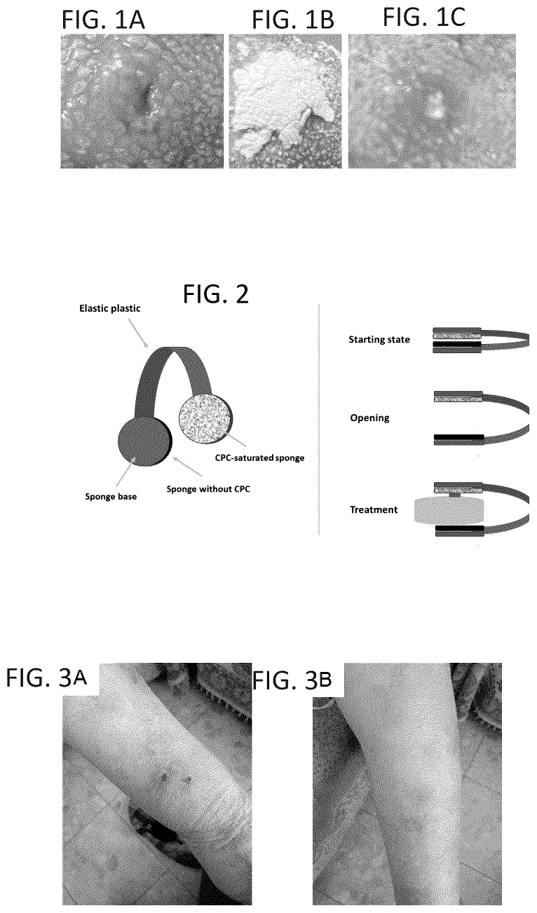

Four cases of aphthae ulcers (FIG. 1A) were treated by applying CLC powder (FIG. 1B) for 15-30 minutes. The subjects reported a numb and painless sensations for a few hours. They could eat and drink normally. When the pain was renewed later it was less intense until it gradually vanished the next day (FIGS. 1A-C). The ulcer completely healed after two days from applying CLC.

Applying CLC powder on aphtha on the tongue or in the mouth is challenging therefore a special device was invented, the device is made of two sponges connected with elastic plastic, that can be clamped. One of the sponges is saturated with CLC powder and is placed directly on the ulcer (FIG. 2). Another option for treatment is using CLC as gel or ointment.

### Example 2

### Repairing Chemotherapy-Induced Leg Ulcers

Chemotherapy is a type of cancer treatment that uses drugs to kill cancer cells. One of the side effects of chemotherapy can be the development of sores or wounds on the legs, also known as leg ulcers. These ulcers are caused by poor circulation in the legs, another side effect of chemotherapy. Symptoms of leg ulcers from chemotherapy may include pain, swelling, redness, etching and discharge. Treatment of leg ulcers typically includes measures to improve circulation, such as compression stockings or exercises to help pump blood through the legs.

Wound dressings, such as hydrocolloid dressings, hydrogel dressings, or alginate dressings, may be applied to the ulcer to help promote healing and reduce the risk of infection. In more severe cases, wound vacuum may be used to remove excess fluid from the wound and promote healing.

The duration of healing varies with the severity of leg ulcers, the overall health of the patient and the response to the treatment. In some cases, these ulcers may take several weeks or even months to heal, with proper treatment and management.

### Materials and Methods

CLC preparation as in Example 1.

### Treatment

A subject (n=1, female, age=78) having chemotherapy-induced leg ulcers was treated with CLC powder (2-3 g). The subject applied the powder directly to the wound once a day for 4 days, covering the entire injured area and its depth.

### Results

CLC powder was used on chemotherapy-induced leg ulcers, which is featured by discontinue of the tissue, itching, skin redness, and inflammation (FIG. 3A). Three days posttreatment the open ulcers were closed showing enhanced tissues recovery with redness and inflammation totally disappearing. After a week normal tissue was observed (FIG. 3B).

### Example 3

### Reducing Pain and Wound Healing Following Hemorrhoids, Anal Fissures

Hemorrhoids, anal fissures are common conditions that affect the rectum and anus. Hemorrhoids, also known as piles, are swollen and inflamed veins in the rectum or anus. They can be internal, inside the rectum or external, around the anus. Fissures, on the other hand, are small tears or cracks in the skin around the anus. Symptoms of both hemorrhoids and anal fissures can include itching, burning, physical and emotional pain, and bleeding during bowel movements. Medical treatment of hemorrhoids and anal fissures depends on the severity of the condition. Mild cases of hemorrhoids may improve within a few days with over-the-counter creams and ointments.

For more severe cases, it may take several weeks or even months for symptoms to improve with medical treatment. In some cases, surgery may be required to remove the hemorrhoids. The recovery time after surgery can vary, but it typically takes several weeks for full healing.

### Materials and Methods

CLC preparation as in Example 1.

### Treatment

Twenty-eight subjects (n=28 adults aged 24-81; 8 male, 20 female), suffering from mild to severe hemorrhoids/fissures were treated with 2-5 g CLC powder. CLC was used in three different forms: as a powder in proctoscope or an applicator; as a gel/ointment or as a suppository of glycerin or Proctoglivenol™.

### Results

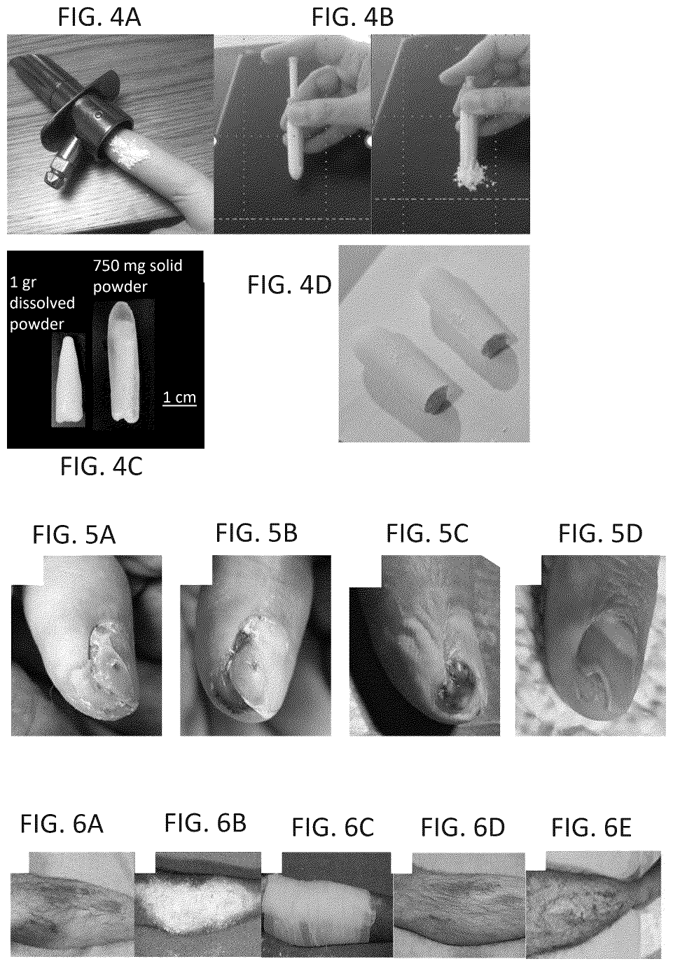

CLC was used to treat 28 cases of mild to severe hemorrhoids/fissure, all of them suffering from enormous pain (scale 8-10 in a 1-10 pain intensity scale). CLC was used in three different forms: as a powder in proctoscope or an applicator; as a gel/ointment or as a suppository of glycerin or Proctoglivenol™ (FIG. 4A-D).

36% of the participants reported significant relief of pain (from 8-10 (pain intensity scale) to 1-2) but temporarily-lasting several hours. 48% of the participants experienced a significant decrease in pain (from 8-10 to 1-2) that lasted for days, while 16% of the participants reported that pain disappeared completely after one treatment (from 8-10 to 0). None of the subjects reported an increase in pain, fibrosis, narrowing of the anal canal, skin itching, allergic reactions, or systemic side effects due to the treatment. Treatment of hemorrhoids/fissure diseases with CLC reduces inflammatory and nociceptive pain, indirectly relaxing muscle tone and facilitating defecation.

### Example 4

### Repairing Non-Bleeding Skin Injuries-Cuts

Skin injuries are a common type of injury that can range from minor cuts and bruises to more severe burns, such as heat, chemicals, and even jellyfish stings.

The body's response to a skin injury can vary depending on the type and severity of the injury. In general, the body's immediate response to a skin injury is pain (many times it involves throbbing pain), which alerts the individual to the presence of the injury. Inflammation and a pain response occur as the skin cells are destroyed. The body then begins the healing process, which involves tissue repair and the formation of new skin cells.

The healing time for skin injury can vary depending on the severity of the injury. Minor injuries may heal within a few days to a week, while more severe ones can take several weeks to months to heal.

### Materials and Methods

CLC preparation as in Example 1.

### Treatment

A subject (n=1, male, 50 years old), with a knife cut penetrated through the nail and cut the finger's tissue, causing skin redness, inflammation and throbbing pain (FIG. 5A). Treatment was provided by application of CLC directly to the wound (2 g). The amount of powder used was selected to cover the entire injured area and its depth, bandaging overnight and an additional dose the next day.

### Results

Applying CLC powder to a skin injury reduces pain immediately and accelerates wound healing and tissue repair. In the reported case once CLC was applied to the cut, the throbbing pain disappeared in seconds (from 8 (in a 1-10 pain intensity scale) to 0). The following day, neither pain nor contamination were reported. Five (FIG. 5B) to eleven days post injury, the healing process was still underway and although the tissue had not fully repaired at this stage, there was no pain, infection, or inflammation present (FIG. 5C). The wound healed and the tissue was repaired after 30 days (FIG. 5D).

### Example 5

### Repairing Skin Injuries-Heat Burns

Heat burn injury affects both the epidermis (outer layer of skin) and the dermis (inner layer of skin). The healing process of a burn wound involves several stages, including inflammation, tissue repair, and the formation of new skin cells. In some cases, the body produces a scar to strengthen the area and prevent further injury.

### Materials and Methods

CLC preparation as in Example 1.

### Treatment

A subject (33 years old, male) suffered a second-degree heat burn injury on the anterior part of the knee, resulting in pain, swelling, and blister formation. The subject began daily treatment of 5 g CLC powder, on the third day following the injury (FIG. 6A). As part of treatment, a layer of CLC powder was applied to the affected area and bandaged covering the entire injured area and its depth (FIGS. 6B and 6C). The bandage was left overnight and the treatment was repeated every day.

### Results

A subject (33 years old, male) suffered a second-degree heat burn injury on the anterior part of the knee, resulting in pain, swelling, and blister formation. The subject began daily treatment with CLC powder on the third day following the injury (FIG. 6A). As part of treatment, a layer of CLC powder was applied to the affected area and bandaged (FIGS. 6B and 6C). The bandage was left overnight and the treatment was repeated every day. Blisters disappeared within a day of treatment (FIG. 6D) and wound healing took approximately two weeks (FIG. 6E). No contamination occurred at any time point.

### Example 6

### Repairing Skin Injuries-Chemical Burns

Jellyfish stings (chemical burn) occur when the tentacles of a jellyfish release stinging cells, or nematocysts, into the skin. The body's immediate reaction to a jellyfish sting is pain and formation of red blisters may form on the skin. The healing time for jellyfish stings can vary depending on the type of jellyfish and the individual's reaction to the sting. In most cases, the blisters may last for 1 to 2 weeks, and itchy skin rashes may appear 1 to 4 weeks after the sting.

### Materials and Methods

CLC preparation as in Example 1.

### Treatment

A subject (n=1. Female, 50 years old) started CLC powder treatment a day post jellyfish (Rhopilema nomadica) sting injury. The subject applied 3 g CLC powder directly to the ulcers, covering the entire injured area and its depth.

### Results

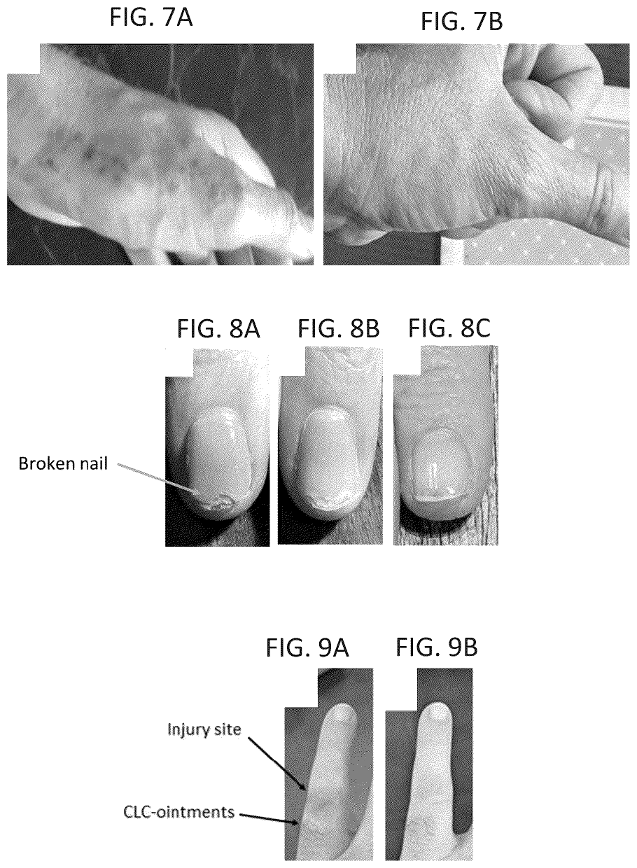

A case of a Jellyfish sting injury featuring blistered skin with red welts, redness, itching and inflammation. The use of CLC powder resulted in a significant reduction in skin redness and complete elimination of blisters within one day of treatment (FIGS. 7A-B). Symptoms were not reported again.

It can be concluded that CLC powder is an effective treatment for skin injuries. It accelerates the healing of burn wounds, quickly eliminates blisters, and serves as an antiseptic without causing side effects (such as allergies, burning sensation or redness).

### Example 7

### Reducing Symptoms of Skin Condition

Acne is a common skin condition characterized by pimples, blackheads, and whiteheads that usually occur on the face, neck, back, and chest. Acne is caused by a combination of factors, including the overproduction of oil in the skin, the accumulation of dead skin cells, and the growth of bacteria. Hormonal changes during puberty, stress, and genetics can also contribute to the development of acne.

CLC preparation as in Example 1.

### Treatment

A subject (13 years old, child) suffered from acne featuring oily and irritated pimples on his face. The child was treated with 0.5-1 g of CLC powder, which covered his pimples overnight.

### Results

A case of a subject with oily and irritated skin characterized by red and swollen, itchy pimples was treated with CLC powder overnight. The following day, the skin was soft and no longer oily, with only small red dots visible but without swelling or edema.

### Example 8

### Nails Strengthening

There are many factors that can cause weak nails and brittle nails, such as nutritional deficiencies: a lack of biotin, iron, fatty acids, and other essential vitamins (B vitamins) and minerals (calcium), hormonal changes, hormonal imbalances during menopause or pregnancy, medical conditions, such as anemia, hypothyroidism, or psoriasis; exposure to chemicals, such as cleaning products; age: as people age; overuse of nail products, etc. Weak nails and brittle nails can lead to discomfort and a painful experience.

### Materials and Methods

CLC preparation as in Example 1.

A mixture of CLC powder and nail polish:
mixing 1 g of CLC powder with 15 ml of Commercial Strengthener. The Commercial Strengthener is composed of a lacquer base, 5% formaldehyde, and 5% calcium carbonate.

### Treatment

A subject (57 years old, female), who suffered from weak and brittle nails for years. Over the past year and a half, her nail condition has worsened since she began undergoing chemotherapy. The subject was treated with a mixture of CLC powder and nail polish. She applied a new layer of CLC-nail polish, for 14 days.

### Results

Treatment of a subject with weak and brittle nails (FIG. 8A) using a mixture of CLC powder and nail polish led to strengthened nails and the recovery of new tissue after just two days (FIG. 8B). After 14 days, the nails had grown normally and were no longer weak or brittle (FIG. 8C).

### Example 9

### Reducing/Eliminating Muscle Spasm/Tendon Inflammation

Back pain is a prevalent problem, affecting nearly 80% of individuals worldwide at some point in their lives. Its causes are diverse and can include strains in muscles or ligaments, herniated or bulging discs, arthritis, scoliosis, and various health conditions. Additionally, it can result from excessive usage, injury, or poor posture. Back pain typically presents as a dull or sharp ache in the lower, middle, or upper back, along with stiffness, muscle spasms or cramps, and restricted movement.

### Materials and Methods

CLC preparation as in Example 1.

### CLC Ointment Preparation:

CLC ointment was prepared by dissolving 4 gr of CLC powder in 25 ml olive oil.

### Treatment

Five subjects (4 male, age 60-65, 1 female, age 82) experienced episodic severe back pain, caused by various health issues, including scoliosis with a pain intensity level of 8-9 out of 10. And 3 subjects (2 male, age 65, 1 female, age 82) suffered from back muscle spasm/tendinitis were treated with 3-5 ml CLC ointment. The CLC ointment treatment was expanded to knee and neck pains (2 ml CLC ointment). Seven subjects (females, ages 60-80) experienced severe pain in their knees and one subject (age=65) had pain in their neck (2 ml CLC ointment).

### Results

CLC ointment, was used to treat subjects with back pain or back muscle spasm/tendinitis, knees and neck pain. Over half of the patients in the first group experienced temporary numbness, which was quickly followed by substantial relief that lasted for several minutes to several days. The pain in the back either partially or completely disappeared, allowing the subjects to return to their daily activities. All of the subjects with muscle back spasms reported immediate elimination of pain, within a few seconds to a minute, after a single treatment.

In over 70% of the subjects, suffered from knee pain, the pain vanished completely. For the rest, the pain was scientifically rated at a reduced level of 2, in a pain severity scale of 1-10, within seconds to a minute.

To sum up, most of the subjects reported feeling a tingling or local numbness after treatment, lasting for a few seconds to minutes, which significantly reduced or even eliminated the pain. This indicates the analgesic effect of CLC treatment.

### Example 10

### Reducing/Eliminating Heat Related Pain

Mechanical hits may cause damage to the body tissues or structures. It can be accompanied by pain, swelling and inflammation, redness, bruising, or restricted movement.

### Materials and Methods

CLC preparation as in Example 9.

### Treatment

A case of mechanical injury on a finger near the joint (1 male, age 63), which included pain, redness, and movement limitation, was treated with 1 ml CLC ointment 8 minutes post-injury.

### Results

The subject reported that the pain vanished after 30 minutes, the redness disappeared, and normal hand movement was reported (FIGS. 9A-B).

### Example 11

### Reducing/Eliminating Itching Sensation

Itching is a sensation which typically occurs when nerve endings in the skin are stimulated. It can be caused by a variety of factors, including insect bites, allergic reactions, and skin conditions such as eczema.

When a person experiences an itching sensation, they may be tempted to scratch the affected area. Scratching can provide temporary relief by interrupting the nerve signals that are causing the itching sensation. However, repeated scratching can break the skin's protective barrier, damage the skin, and create wounds. This can introduce bacteria and other microorganisms into the body, increasing the risk of infection.

### Materials and Methods

CLC preparation as in Example 1.

### Treatment

One case of repeated itching sensation that caused wounds between the toes (men, age 54) was treated with 0.5 g of CLC powder overnight.

### Results

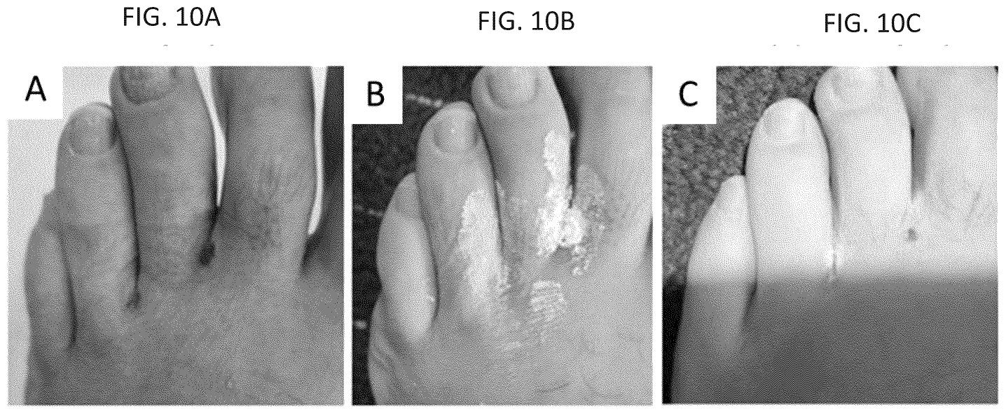

The subject was treated with a single dose of CLC powder overnight. The itching sensation was abolished after the treatment (FIGS. 10A-C).

### Example 12

### Treatment of Gum Infection

Gum infection, also known as periodontitis, is a condition where the gums and the surrounding tissues of the teeth become inflamed and infected. This occurs as a result of plaque buildup on the teeth that causes irritation to the gums. Over time, the infection can spread deep into the gums, leading to destruction of the surrounding tissues and bones that support the teeth. Common symptoms of gum infection include red, swollen gums, bleeding while brushing or flossing, bad breath, and loose teeth. Treatment typically involves deep cleaning of the affected area, along with antibiotics to clear the infection. In severe cases, surgery may be required to remove the damaged tissue and repair the affected area.

### Materials and Methods

CLC preparation as in Example 1.

### Treatment

A 57-year-old female presented with gum damage on both sides of her mouth reported a severe pain rated at 8 out of 10. Treatment involved brushing the affected gum area with 1 g of CLC powder, once.

### Results

According to the subject, brushing with powder significantly reduced both gum damage and pain. The pain, which was previously rated as an 8 out of 10 on a pain scale, disappeared completely within seconds after brushing.

### Example 13

### Treatment of Glioblastoma

Glioblastoma is the most aggressive and malignant primary brain tumor in adults. It is characterized by its rapid growth and tendency to invade nearby healthy brain tissue.

### Materials and Methods

CLC preparation as in Example 1.

### Epsilon CPC (E-CPC) Preparation:

Same as CLC preparation (example 1), but instead of L-Lysine, Poly-ε-Lysine solution is used (Cat. FP14985, Biosynth) for the generation of Calcium-Poly-ε-Lysine-citrate, termed E-CPC. In contrast to lysine monomers, which can bind calcium ions through its open positively charged residue, in Poly-ε-Lysine these residues are blocked due to their interaction with neighboring residues. Hence, differences in effectiveness of CLC vs E-CPC may be attributed to the lysine portion of CLC. In addition, Poly-ε-Lysine has a strong antibacterial activity and may improve the effectiveness of the E-CLC to heal wounds. It is therefore of great importance to check the level of its multi-modality by assessing its effect on glioblastoma cells.

Three experiments were conducted in which the survival rate of glioblastoma (A172) cells in culture was compared between CLC or E-CPC treated for 5 days, vs untreated cells.

### Results

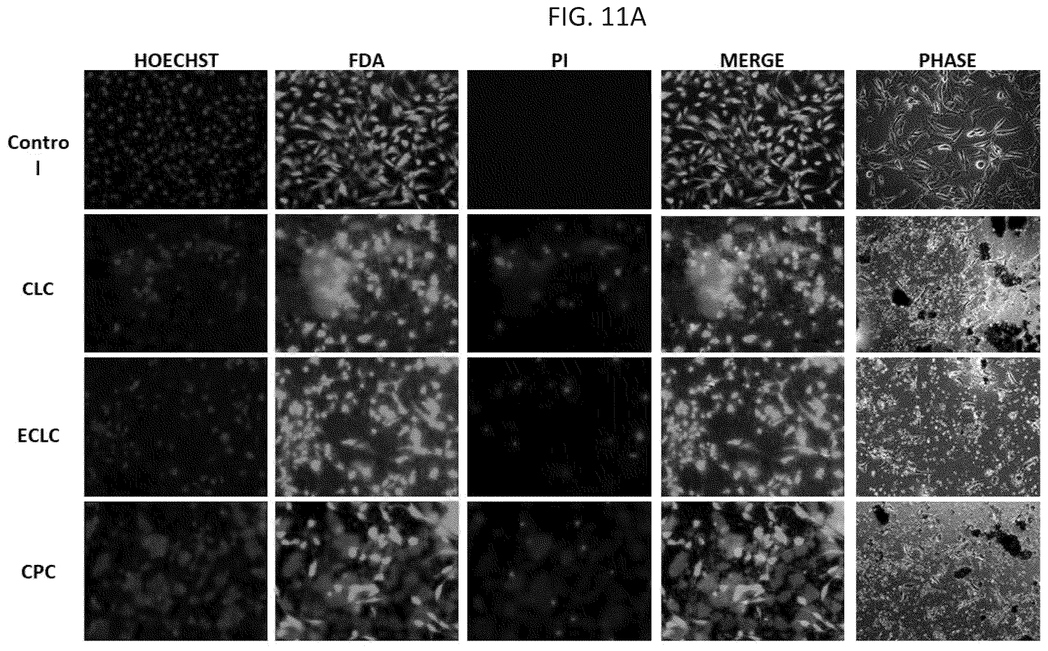

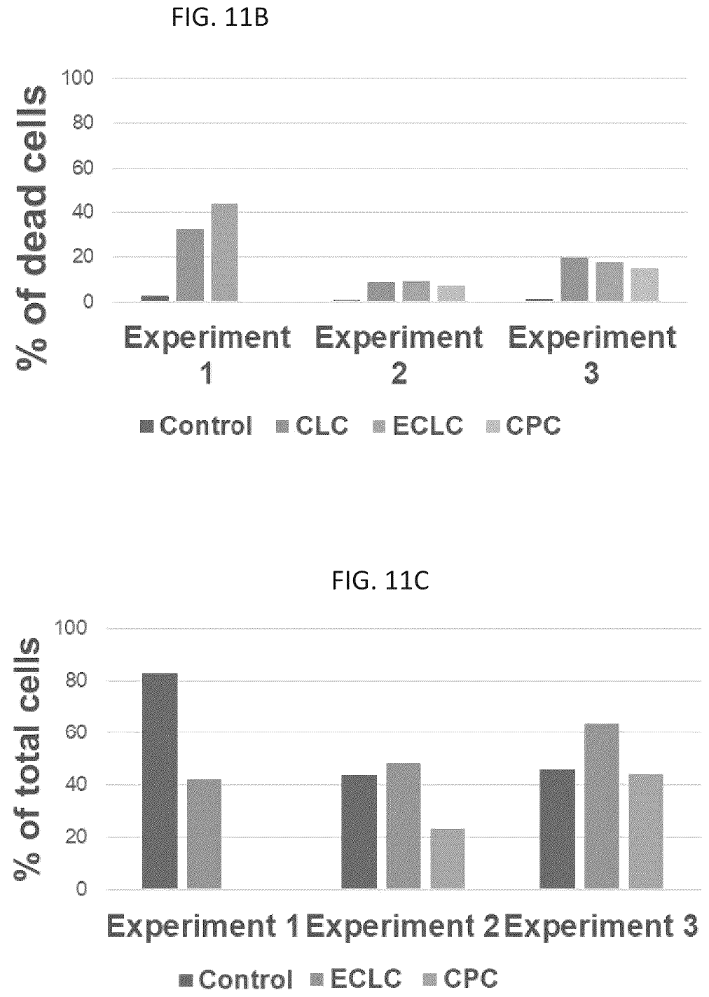

The survival rate of glioblastoma (A172) cells in culture was compared between CLC or E-CPC treated cells. The results show that the total number of cells in the treated culture was reduced by approximately 2 folds than the control, which was manifested by lower cell confluency in the treated coverslips compared to control. Moreover, out of the residing cells, a 2-3 folds elevation in the percentage of dead cells was detected in the treated vs the non-treated cultures (FIGS. 11A-C).

### Example 14

### CLC and E-CLC Act in a Dose Dependent Manner and Specifically Affect Glioblastoma Cells Survival, Morphology and Metabolic Activity

### Materials and Methods

### Preparation of CLC/for Treatments as in Example 1.

Treatment (CLC): For CLC treatment, 100 μL of cell media containing the required concentration of cells were first seeded onto sterile Coverslip placed in 24-well plates and incubated overnight. Next day, the old cell medium was removed and CLC was mixed in cell culture medium (500 μL) and added to respective wells, at the indicated concentrations.

### Human Glioblastoma A-172 Cell Culture:

The A-172 cells were routinely grown in the glucose DMEM media (Cat. D5796 Sigma-Aldrich) supplemented with 10% FBS and a 1% antibiotic-antimycotic solution. The cells were maintained in a 10% CO₂ incubator under humidified conditions. For immunofluorescence experiments, cells were trypsinized, and seeded on the Coverslip (80,000 cells/mL). For MTT assay, cells (20,000 cells/mL) were seeded directly to 96 well plates.

### PI-FDA- and Hoechst Staining:

A172 cells were seeded onto desired Coverslip and placed in 24 well plates at a seeding density of 8000 cells/Coverslip. On the day of termination, sample Coverslip were then stained with 1% Propidium Iodide (PI) 2 μg/ml (Cat. P4864, Sigma-Aldrich), 0.16% Fluorescein Diacetate (FDA) 5 μg/ml (Cat. F7378, Sigma-Aldrich) and 0.1% Hoechst 1 mg/ml (Cat. B2261, Sigma-Aldrich) for 5 minutes at RT to visualize dead cells, live cells and cell nuclei, respectively. The dyes were then rinsed twice with PBS (1×) and were instantly visualized through a fluorescent microscope.

Percentage of cell number reduction=[{(total cells in control−total cells in treated)/total cells in control}*100]

### Immunofluorescent Staining:

Primary cells will be cultured onto sterile poly-D-lysine-coated Coverslip placed in 24 well plates at a seeding density of 40,000 cells/Coverslip and uncoated treatment will be given. They will be incubated for 3 and 5 days respectively. Samples Coverslip will be then fixed with 4% Paraformaldehyde (Cat. P6148, Sigma-Aldrich) for 10 minutes, permeabilized with 0.25% TritonX100 (Cat. H5142, Promega) for 5 minutes, and blocked with 3% inactivated Normal Goat Serum (Cat. 04-009-1A, Biological Industries) for 1 hour. All the procedures above will be performed at room temperature. The samples will then be incubated overnight at 4° C. with unconjugated primary antibodies like, Anti-Glial Fibrillary Acidic Protein (GFAP) (Cat. Ab7260, Abcam, 1:500) for glial cells, Anti-Microtubule Associated Protein 2 (MAP2) (Cat. Ab11267, Abcam, 1:400) for visualization of dendrites. The next day, the samples will be treated for 1 hour at RT with secondary antibodies, Alexa488 conjugated Goat Anti-Mouse (Cat. A11001, Life Technologies) and Alexa555 conjugated Goat Anti-Rabbit (Cat. A21429, Life Technologies). 4′,6-Diamidine-2′-phenylindole dihydrochloride (DAPI) (Cat. D9564, Sigma-Aldrich) staining will then be performed for 15 minutes at RT, followed by washing 3 times with PBS (1×). Fluoromount (Cat. F4680, Sigma-Aldrich) will be used as mounting solution to which 2.5% of 1,4-diazabicyclo[2.2.2]octane (DABCO) will be added to improve the lifetime of the samples.

### MTT Assay:

Cells were seeded in 96 well plates at the initial seeding density of 2×103 cells per well. They were allowed to attach to the plate for 24 h followed by treatment with different concentrations (0.04, 0.2, 1, 5 and 10 mg/mL) of CLC dissolved in cell culture media for the next 3 and 5 days respectively. The effect of CLC on cell viability was examined by measuring the ability of cells to convert yellow dye MTT to purple formazan crystals. These crystals, after decanting the culture media, were solubilized using DMSO. The absorbance of colored formazan crystals was measured at 570 nm using the Fluostar optima (BMG Labtech, Germany) multiwell plate reader. The percentage of cell reduction was calculated by the following formula:

Percentage of inhibition={(OD of control−OD of treated)/OD of control}*100.

### Results:

### Dose Response of Different Concentrations of CLC on A172 Cells:

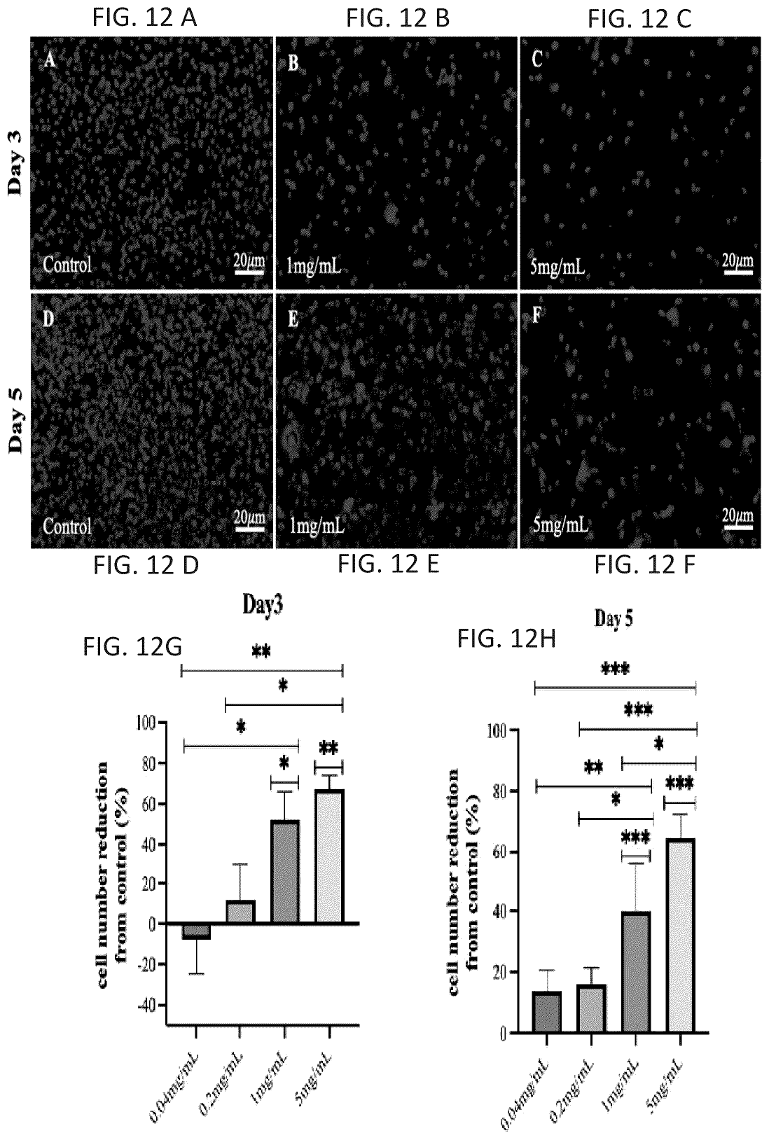

A172 cells were treated with increasing concentrations of CLC from 0.04 mg/mL to 5 mg/mL. By the end of day 3 and day 5, the Hoechst images showed a sharp decrease in total cell numbers in the treated group with respect to the untreated control, in all concentrations tested (FIGS. 12A-F). However, differences in the extent of reduction among the various concentrations were detected, with 1 mg/mL showing ˜50% (3-day treatment) and ˜40% (5-day treatment) whereas 5 mg/mL showed ˜60% cell number reduction after both 3 and 5 days of treatment (FIGS. 12G-H).

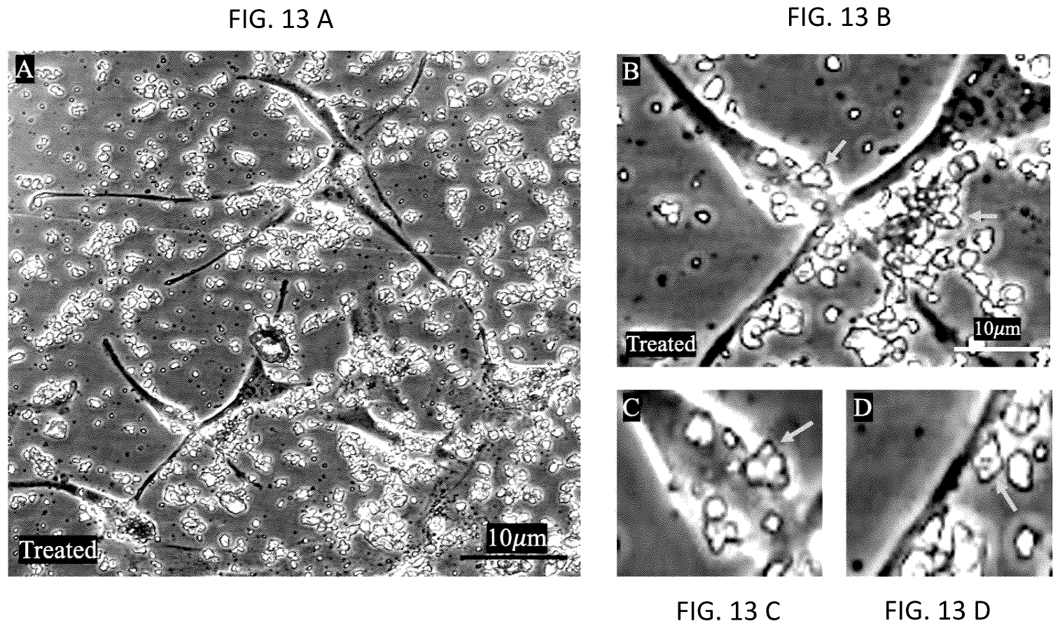

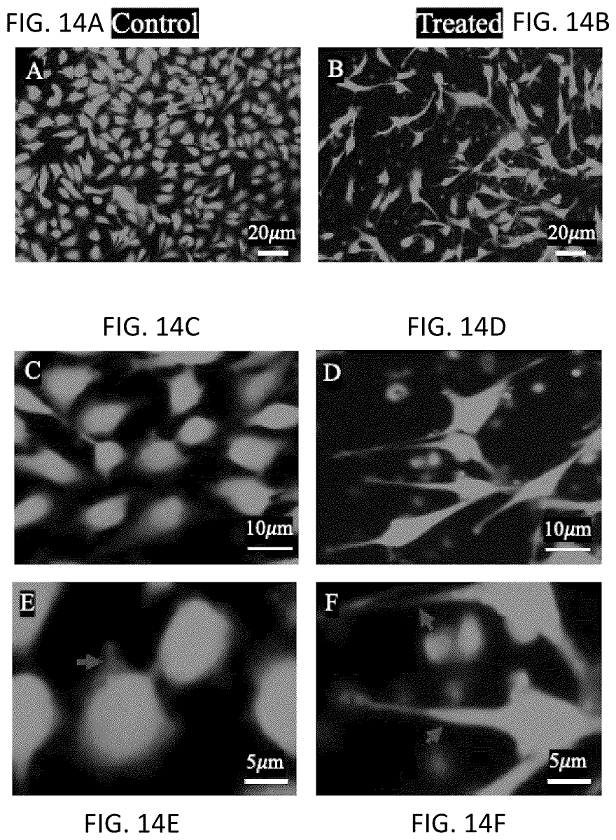

To understand if CLC physically associates with A172 cells, treatment was performed with 5 mg/mL of CLC for 5 days. CLC was found to be in close proximation to the cells and frequently in tight association, as shown in FIGS. 13A-D. CLC particles were found attached to cell bodies and cell processes.

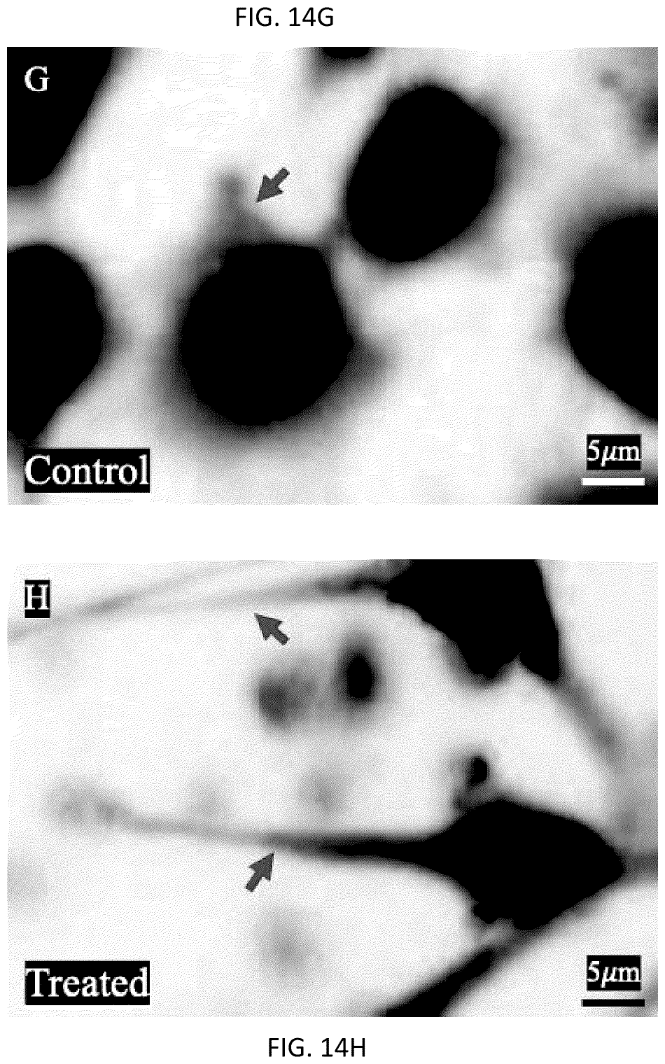

To check if CLC affects A172 cell morphology, cell bodies and processes were fluorescently labeled with FDA. As shown in FIGS. 14A-H, on the 5th day of treatment, cells became thinner than the control cells characterized by showed longer processes (marked in red arrows).

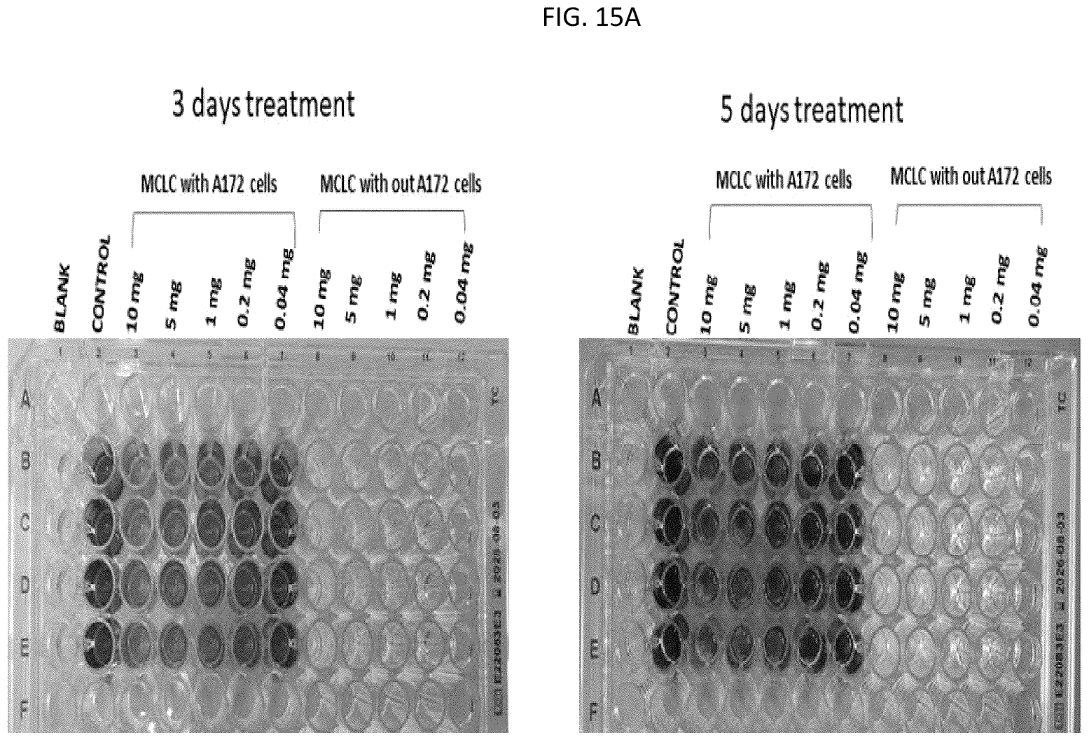

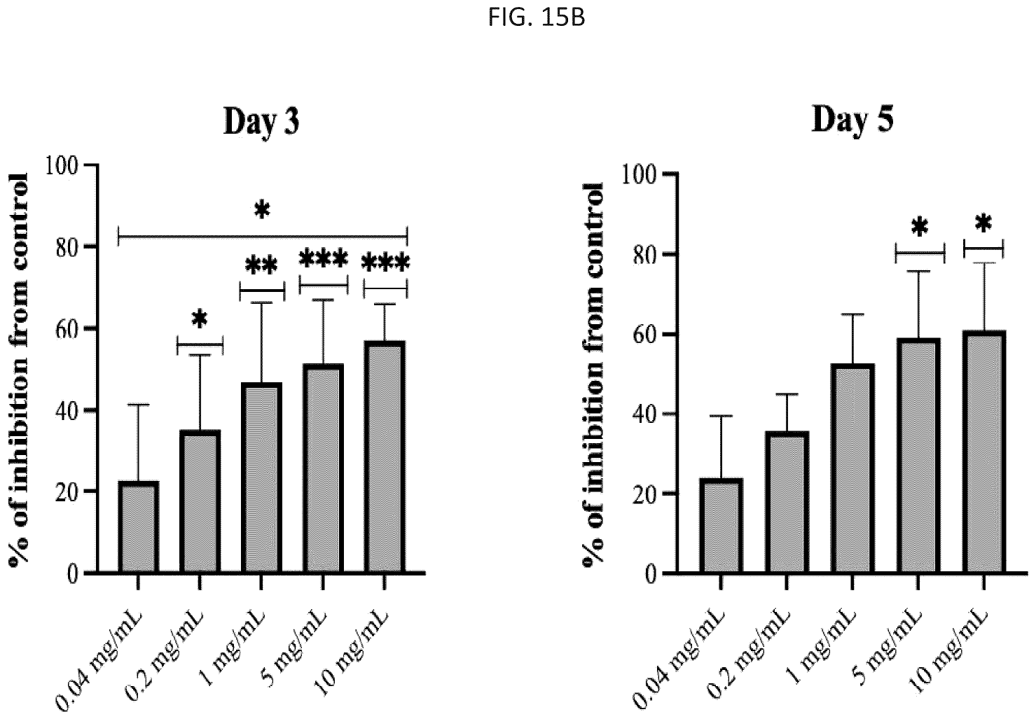

To further test the effect of CLC on cell cytotoxicity, an MTT assay was performed on A172 cells, keeping untreated A172 cells as control. The treatments showed a decreasing color intensity with increasing concentrations of CLC. The MTT dye used showed no color change in the wells containing only CLC without cells, confirming that the CLC grains do not absorb the MTT dye (FIG. 15A). The percentage of cell inhibition was calculated which was in the range of 45-60% for concentrations 1-10 mg/mL at the end of 3 days of treatment. Even a CLC concentration as low as 0.02 mg/mL showed significant percentage of inhibition (˜35%) when treated for 3 days (FIG. 15B). At the end of day 5 of CLC treatment, 5 mg/mL and 10 mg/ml of treatments showed significant cell proliferation inhibition, approximately 60% (FIG. 15B).

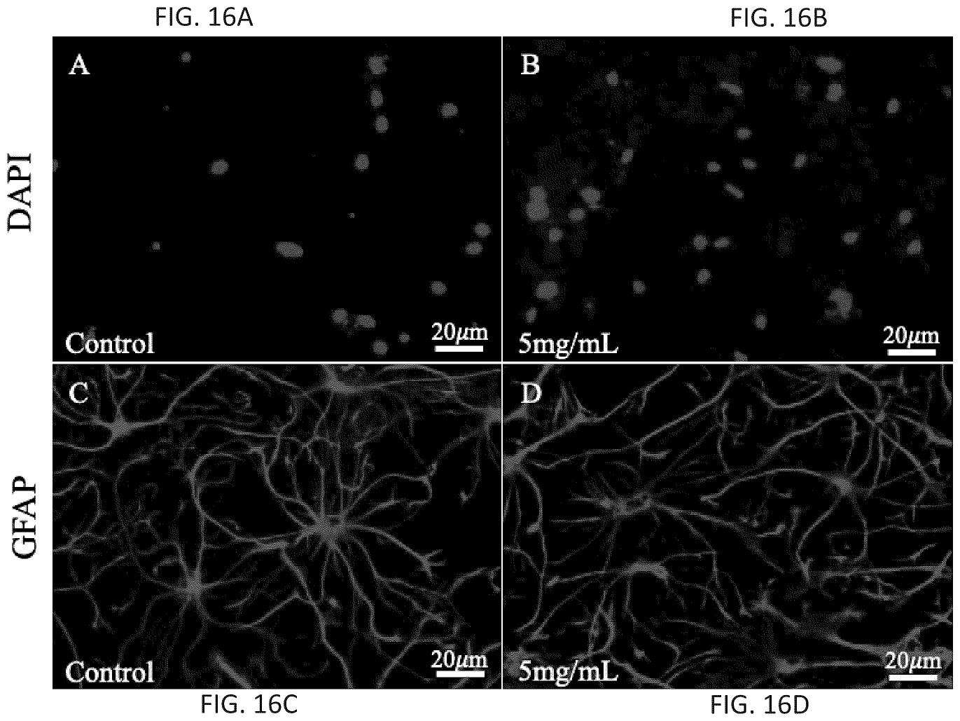

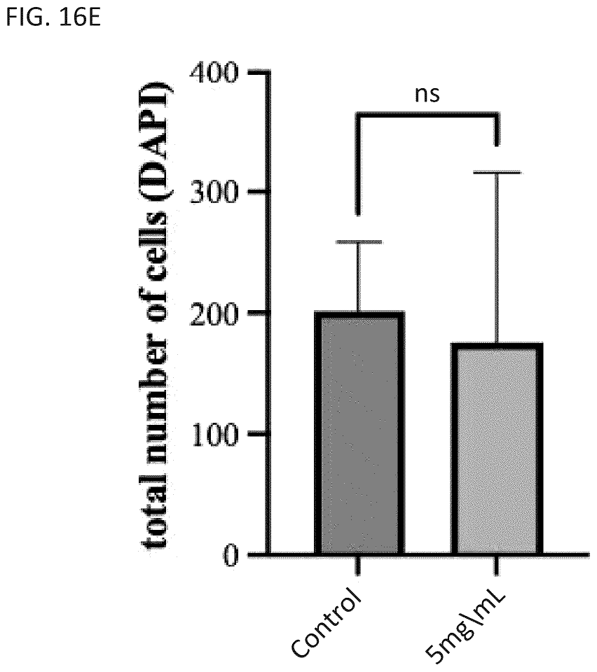

Finally, to check if CLC cytotoxic activity is specific to glioblastoma cells, as opposed to healthy astrocytes, astrocytes where isolated from rat hippocampi and grown in culture for 2 weeks and then treated with 5 mg/mL of CLC for 5 days. No significant difference in total cell number or in cell shape between the untreated and treated cells was detected (FIGS. 16A-E and 17A-E). These results show that the cytotoxic effect is specific to the glioblastoma cells.

### Example 15

### CLC as Anti-Bacterial Agents

CLC preparation as in Example 1.

### Treatment

Testing the Effect of CLC Treatment on Staph. Aureus Total Number and Viability:

Staph. Aureus bacteria were seeded on Petri dishes containing Nutrient Agar. In the treatment plates, 10 mg of CLC powder was placed in certain places on the plates, above the bacteria. Control (without CLC powder) and treatment plates were then incubated at 37° C. overnight. The next day, the treated areas and areas from the control plates were punctured using a 6 mm biopsy punch. The samples were transferred to 1.5 ml test tubes and mixed using a vortex in order to release all the bacteria from the agar. After sedimentation of the agar, the supernatant was transferred into a new test tube. The test tubes were centrifuged at a speed of 3,000 g for the sedimentation of the bacteria and the supernatant was discarded. A staining solution containing 0.1% Hoechst for staining bacterial chromatin, and 1% Propidium Iodide for staining dead bacteria, was added to the test tubes. The bacteria were incubated with the dye solution for 4 minutes and then centrifuged at 3,000 g two more times, each time being suspended in fresh PBSX1 for washing. 200 μl were taken from the bacterial solutions, transferred to a plate with a glass bottom and taken for microscopic examination.

### CLC on Back Wound in Mice (2 Days)

Two months-old ICR mice were divided into two separate groups, control group and CLC-treated group. The mice were anesthetized using Ketamine and Xylazine solution in a ratio of 1:3, respectively, that was injected to them intraperitoneally. The backs of the anesthetized mice were shaved and a 6 mm-diameter biopsy punch was performed on their shaved skin, removing the skin and the underlying muscle tissue. A silicone ring with an outer diameter of 10 mm and an inner diameter of 7 mm was sutured around the wound to avoid its spontaneous contraction. The CLC-treated mice, 30 mg of CLC powder was applied to the injured area, while the control mice remained untreated. All wounds were covered with a plaster, and the mice were treated by a subcutaneous injection of Carprofen against pain, and 2 ml of saline solution 0.9%. When recovered from anesthesia, were returned to the animal house. 24 hours post-procedure, a swab was taken from the wounds and seeded on a Petri dish containing Nutrient Agar. The dishes were then incubated overnight at 37° C., and then observed for general bacterial growth.

### Results

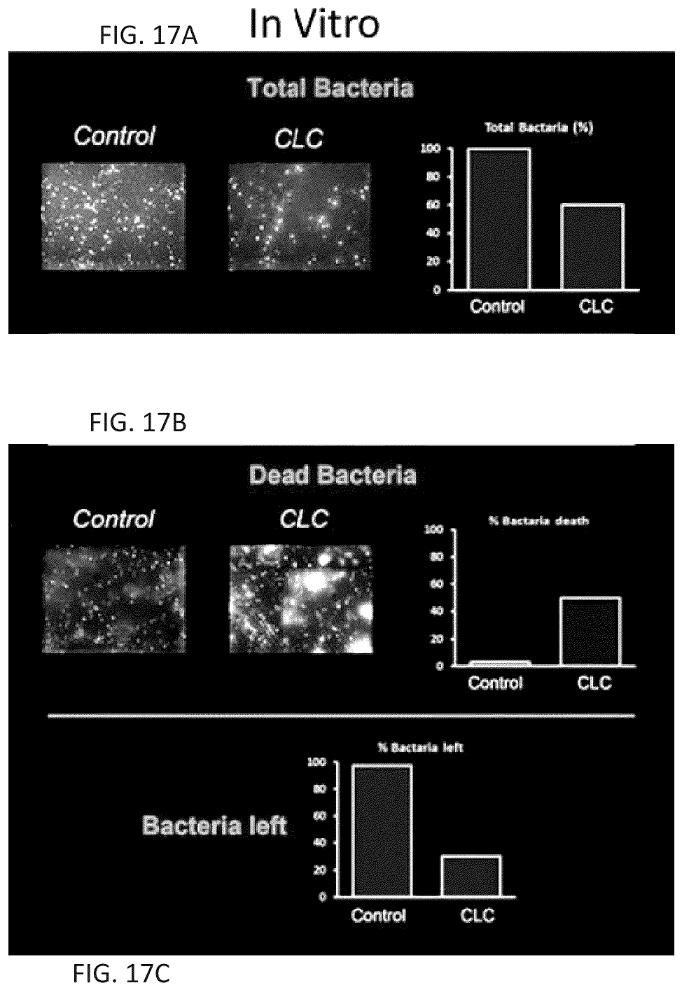

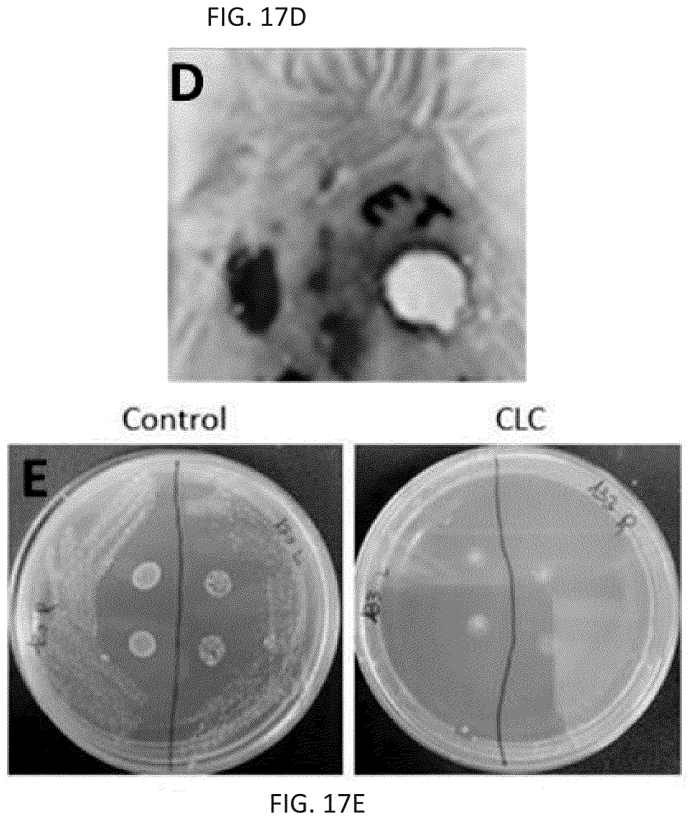

Bacterial chromatin staining revealed that after contact between the Agar-seeded bacteria and the CLC for overnight, there was a reduction of 40% in total number of Staph. Aureus bacteria (FIG. 17A). In addition, among the remained bacteria, an elevation of the percentage of PI-positive bacteria was observed, from 2% spontaneous death among control to 50% death after CLC treatment (FIG. 17B). These results may indicate that the negative effect of CLC is mutual on bacterial division, as well as on their viability. Reduction of the dead bacteria from the total number of bacteria revealed that after CLC treatment the general presence of live bacteria was reduced by 70% (FIG. 17C). 24 h-old wounds that were examined for general bacterial development clearly showed a decrease in bacterial growth on Nutrient Agar Petri dishes that were incubated for 24 hours at 37° C. (FIGS. 17 D-E).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating a medical condition selected from the group consisting of aphtha, chemotherapy-induced wound, acne, hemorrhoids, a fissure, itching, a chemical-induced burn or a heat-induced burn, a brittle nail, muscle spasm, tendon inflammation, gum inflammation, glioblastoma and a bacterially contaminated wound in a subject in need thereof, the method comprising administering to the subject an effective amount of a composite material comprising a calcium carbonate-containing material, and an associating moiety being associated with said calcium carbonate-containing material, thereby treating the medical condition in the subject.

2. The method of claim 1, wherein said subject is a human subject.

3. The method of claim 1, wherein said subject is a non-human subject.

4. The method of claim 1, wherein said administering comprises topical administration or wherein said composite of formulated for topical administration.

5. The method of claim 1, wherein said effective amount comprises multiple administrations, optionally wherein said multiple administrations are affected over a period of at least 7 days.

6. The method of claim 1, wherein said composite is formulated in a form of a powder, a gel, a spray, a foam, a mousse, an ointment, a paste, a lotion, a gauze, a wound dressing, a suspension, an adhesive bandage, a non-adhesive bandage, a wipe, a gauze, a pad, an enema, a suppository and a sponge.

7. The method of claim 1, wherein said composite further comprises a citrate in chemical association with the calcium carbonate, and/or with the association moiety and/or wherein said associating moiety is a positively-charged moiety at physiological pH.

8. The method of claim 1, wherein said association moiety is a polymeric moiety, optionally wherein said association moiety has a molecular weight in a range of 10 to 100 kDa.

9. The method of claim 1, wherein said association moiety is a biocompatible moiety.

10. The method of claim 1, wherein said association moiety is a polypeptide, optionally wherein said polypeptide comprises at least one amino acid residue that is positively charged at physiological pH.

11. The method of claim 10, wherein said polypeptide essentially consists of amino acid residues that are positively charged at physiological pH, optionally:
wherein said polypeptide is or comprises a polylysine, optionally wherein said polylysine is selected poly-D-lysine, poly-L-lysine and poly-s-lysine.

12. The method of claim 10, wherein said polypeptide is or comprises collagen.

13. The method of claim 1, wherein said association moiety is or comprises lysine, optionally wherein said lysine is selected from L-lysine and D-lysine.

14. The method of claim 1, wherein the calcium carbonate-containing material comprises crystalline calcium carbonate, optionally wherein said calcium carbonate-containing material comprises a coral exoskeleton.

15. The method of claim 1, wherein the calcium carbonate-containing material comprises amorphous calcium carbonate (ACC) or calcite.

16. The method of claim 1, wherein said calcium carbonate-containing material is a particulate material, optionally wherein said particulate material comprises particles having an average particle diameter in the range of from 0.01 micron to 10 millimeter, or from 0.01 micron to 1 millimeter, or from 0.01 micron to 500 microns, or from 0.1 microns to 500 microns, or from 1 micron to 500 microns, or from 5.0 microns to 500 microns;
wherein said particulate material comprises particles having an average particle diameter in the range of from 0.01 micron to 100 microns, or from 0.01 microns to 50 microns; or
wherein said particulate material comprises particles having an average diameter in the range of from 20 nanometer to 10 millimeter, or from 100 nanometer to 1 millimeter.

17. The method of claim 7, wherein a weight ratio of said citrate and said calcium carbonate-containing material ranges from 10:1 to 1:10, or from 5:1 to 1:5; or, wherein a weight ratio of said association moiety and said calcium carbonate-containing material ranges from 5000:1 to 250:1.

18. The method of claim 1, further comprising administering a swelling polymeric moiety, optionally wherein said swelling polymeric moiety is selected from alginate, chitosan, collagen and a poly(alkylene glycol).
